(19) United States
(12) Patent Application Publication
(10) Pub. No.: US 2025/0128043 A1
(43) Pub. Date: Apr. 24, 2025

(54) FLUID MANAGEMENT CONSOLE AND SYSTEM

(71) Applicant: White Swell Medical Ltd, Kibbutz Shefayim (IL)

(72) Inventors: Paul Frehill, Kibbutz Shefayim (IL); Ronan Keating, Galway (IE); Darren Craven, Kibbutz Shefayim (IL); Sagi Raz, Tel-Aviv (IL); Eamon Brady, Galway (IE)

(21) Appl. No.: 18/832,982

(22) PCT Filed: Feb. 3, 2023
(86) PCT No.: PCT/IB2023/000069

§ 371 Date: Jul. 25, 2024

Related U.S. Application Data
(60) Provisional application No. 63/306,248, filed on Feb. 3, 2022.

(51) Int. Cl. A61M 60/13; A61M 60/216; A61M 60/408; A61M 60/554; A61M 60/802

(52) U.S. Cl. CPC A61M 60/13; A61M 60/216; A61M 60/408; A61M 60/554; A61M 60/802; A61M 2205/3553

(57) ABSTRACT

The invention provides systems and methods for controlling the operation of intravascular catheters to treat fluid management disorders such as acute decompensated heart failure, chronic heart failure, ascites, lymphedema, chronic kidney disease, cardiac insufficiency, cardiac value regurgitation, or plural effusions. In particular, the invention provides a console, for use in a clinical setting, where the console controls operation of an intravascular catheter. The console includes a controller with treatment logic and a connected hub. The hub has a connection point to which an intravascular catheter can be connected. When the intravascular catheter is connected to the hub, the controller executes instructions and operates devices on the catheter to relieve the fluid management disorder.

### FIELD OF THE INVENTION

The present invention is directed to a fluid management system designed for treating fluid management disorders in a patient, and related components and methods. In particular, the present invention is directed to sensors and hardware used in intravascular catheters for monitoring and/or managing pressure within a blood vessel.

### SUMMARY OF THE INVENTION

The invention provides systems and methods for controlling the operation of intravascular catheters to treat fluid management disorders such as acute decompensated heart failure, chronic heart failure, ascites, lymphedema, chronic kidney disease, cardiac insufficiency, cardiac value regurgitation, or plural effusions. In particular, the invention provides a console, for use in a clinical setting, where the console controls operation of an intravascular catheter. The console includes a controller with treatment logic and a connected hub. The hub has a connection point to which an intravascular catheter can be connected. When the intravascular catheter is connected to the hub, the controller executes instructions and operates devices on the catheter to relieve the fluid management disorder.

For example, the catheter may include a flow restrictor such as a balloon, or a pump such as an impeller. The console system can take measurements, such as intravascular blood pressure measurements, and execute instructions to operate the catheter. In particular, in preferred embodiments, the system is operable to calculate and use an offset that ensures that corrected fluid pressure values with great precision and accuracy are shown to a physician and/or used in operating the catheter.

Preferably, the console operates the intravascular catheter using intravascular measurements and optionally the offset to treat fluid conditions that impact interstitial (e.g., extravascular) and/or microcirculatory fluid management. To ensure the physician has the most precise and accurate fluid pressure information, the system uses stored correction data (such as calibration data and or thermal sensitivity data and/or drift characteristic data), intracorporeal and/or ambient sensors, and/or calibration/correction instruction programming to determine an “offset” and correct/adjust the direct pressure measurements to more accurate and precise “true” values, useful to precisely guide therapy. The calculated offset(s) are useful as “correction factors”. In one illustrative example, a blood pressure sensor in a jugular may return a reading of 5 mm Hg. But the controller (in the console) uses the correction data and logic (e.g., ambient pressure sensor, stored calibration data, thermal correction data, etc.) to determine and apply the “offset” so that the console can display to the physician that “true” blood pressure in the jugular is, in fact, 8 mm Hg, with significant implications for lymphatic return.

The offset may be calculated using such data as thermal sensitivity data, calibration data, ambient temperature, drift characteristic data or pressure measurements. The controller receives a baseline pressure reading and uses the offset to calculate a corrected “true” fluid pressure in the patient. This precise and accurate “true” pressure is used to inform a physician and/or operate the catheter and relieve the fluid disorder.

In some embodiments, for a patient undergoing acute decompensated heart failure, the system can operate the catheter to create a low pressure region in a venous angle at an outlet of a lymphatic duct. Such embodiments illustrate that consoles of the system include programming logic and data that allow intravascular or circulatory measurements and operations to address interstitial and microfluidic conditions. That is, systems and methods of invention that bridge a gap between intravascular treatments and fluid management as a whole. Bodily fluid systems naturally involve a variety of fluidic systems beyond and adjacent blood vessels, including lymphatic systems with lymphatic vessels and ducts as well as interstitial fluids that play vital health roles outside of vessels and ducts. Systems and methods of the invention use consoles with programming logic and additional data to calculate offsets and aid physicians in treating fluid management disorders holistically, to correctly and appropriately treat aspects of fluid management disorder that reach beyond the principle circulatory system.

Systems and methods of the invention are operable to control a pump, such as an impeller disposed on an intravascular catheter. The impeller may be driven by a motor, which may be at the impeller or in a connected hub. A controller of a console provides instructions and programming logic controlling operation of the pump.

For example, the pump may be controlled using pressure measurement feedback to adjust the pump operation until a target pressure is achieved. Additionally, systems and methods of the invention may include a variety of systems and features for safe operation of intravascular catheters. In an example, a controller (e.g., in a console) of the invention may use a determined corrected pressure measurement to operate a pump (e.g., impeller) while also limiting operation to a safe speed (flow volume). In another example, systems and methods of the invention use temperature and/or pressure measurements to avoid operating an impeller under conditions that promote hemolysis (e.g., shut down or slow down impeller at critical temperature/pressure combinations). Some embodiments detect evidence of the beginning of hemolysis via evidence of impeller resistance or reduced rotational speed, where the system can display a caution on a display of the console or change operation, e.g., change operation of the impeller. Programming logic and instructions in the controller thus aid in controlling operation of a pump to effectively treat fluid management conditions and also keep the entire system operating within safety parameters.

Various aspects of the present invention are directed to systems and methods for conducting a fluid management therapy that effectively monitors pressure during the fluid management procedure and reliably treats fluid management disorders in a patient.

In one aspect, the present invention provides a method of treating a patient with fluid management disorder using an intravascular catheter having pressure sensors. The method includes the steps of
(a) providing the intravascular catheter with a first pressure sensor and a second pressure sensor,
(b) providing thermal sensitivity and/or drift characteristic data and calibration data for the first pressure sensor,
(c) reading pressure data generated by the first and second pressure sensors in an ambient environment,
(d) inserting the intravascular catheter into a blood vessel and reading pressure data generated by the first pressure sensor inside the vessel,
(e) calculating an offset for the catheter based on difference between pressure data generated by first and second pressure sensors, the thermal sensitivity data and/or drift characteristic data and the calibration data of the first pressure sensor,
(f) subtracting the offset from step (e) from the pressure data generated by the first pressure sensor inside the vessel from step (d) to derive a measurement of actual pressure in the vessel, and
(g) transmitting energy through the catheter based on the derived pressure measurement in the vessel.

In a second aspect, the present invention provides a method for monitoring pressure during a fluid management procedure using an intravascular catheter having pressure sensors. The method for monitoring includes the steps of
(a) providing the catheter with a pressure sensor,
(b) providing thermal sensitivity and/or drift characteristic data and calibration data for the sensor,
(c) reading the thermal sensitivity and/or drift characteristic data and calibration data of the sensor,
(d) reading ambient parameters in the procedure room,
(e) inserting the catheter into a blood vessel and calculating an offset for the catheter inside the vessel, and
(f) transmitting energy through the catheter based on the sensor data.

In a third aspect, the present invention provides a method for restricting fluid flow in a blood vessel using an intravascular catheter with a pressure sensor and a restrictor. The method for restricting fluid flow includes the steps of
(a) providing the intravascular catheter having a pressure sensor and a mounted restrictor,
(b) displaying data generated by the sensor on a display screen connected to the catheter by a controller,
(c) inserting the catheter into a blood vessel of a patient and measuring the pressure data generated by the pressure sensor,
(d) filtering the measured data at a selected permeability and displaying the filtered data on the display screen,
(e) expanding the restrictor while monitoring the filtered data, and
(f) detecting the point at which the restrictor contacts the vessel wall from the trend line of the filtered data.

In a fourth aspect, the present invention provides a system for pumping body fluids, the system comprises a pump assembly designed for deploying inside a human body, a controller with a microprocessor and a software designed to operate the pump assembly, a motor configured to drive a pumping element of the pump assembly.

For example, in one aspect the present invention provides a motor control system for pumping body fluids during an intravascular fluid management procedure. The system Includes:
(a) a pump assembly configured for deployment inside a patient,
(b) a motor configured to drive a pumping element of the pump assembly,
(c) a hardware controller comprising a microprocessor and a software. The software is configured with the hardware controller to operate the pump assembly. The hardware controller comprises a primary control circuit, a secondary control circuit, a motor operation detection element, and a switch element, such that the motor operation detection element is configured to generate an electrical output proportional to an operative parameter of the motor, and the switch element is configured to switch the operation of the motor from the first circuit to the second circuit when the operative parameter exceeds a predefined limit or the primary system fails.

In one embodiment, the pump assembly comprise a blood pump. The pump assembly Includes, in some embodiments, a housing encasing the pumping element. The pumping element, in some embodiments, includes an Impeller.

In some embodiments, the switch element comprises a plurality of switch states. In examples, the switch states comprises a first state and a second state. During the first state, in some embodiments, electron flow between the primary control circuit and motor is facilitated. In other embodiments, during the first state, electron flow between the primary control circuit and the motor is blocked. During the second state, in some embodiments, electron flow between the secondary control circuit and motor is facilitated. Likewise, in other embodiments, during the first state, electron flow between the primary control circuit and the motor is blocked. Further, in some embodiments, the plurality of switch states comprises a third state. Switching the switch element from the first state to the second state causes the motor to operate at a preset rotation speed limit, in some embodiments. In some embodiments, switching the switch element from the first state to the second state causes the motor to operate at a preset lower rotation speed limit. Further, In embodiments, switching the switch element from the first state to the second state causes the motor to operate at a preset standby rotation speed limit.

The hardware controller, in some embodiments, further comprises a current detection element configured to generate an electrical output proportional to the current detection by the current detection element. In some embodiments, the hardware controller further comprises a motor torque detection element configured to generate an electrical output proportional to the torque detected by the torque detection element. The software provides a computational operation on the electrical output of the motor torque detection element, in some embodiments. For example, the computational operation produces a computational output, which is used by the software to manage safe operation of the pump. In some embodiments, the manage safe operation of the pump includes
(i) displaying a warning to the physician,
(ii) reducing or increasing the speed of the motor,
(iii) flushing a component of the pump assembly, and
(iv) terminating the treatment procedure.

Further, in some embodiments, the software provides a computational operation on the electrical output of the current detection element. The computational operation produces a computational output, which is used by the software to manage safe operation of the pump. Thus, In some embodiments, the manage safe operation of the pump includes
(i) displaying a warning to the physician,
(ii) reducing or increasing the speed of the motor,
(iii) flushing a component of the pump assembly, and
(iv) terminating the treatment procedure.

In some embodiments of systems of the invention, the manage safe operation of the pump includes
(i) stopping or reducing the speed of the pump when current/torque and/or speed limits are exceeded,
(ii) running the pump at a low fixed speed when a low speed is detected or a system and/or software failure is detected

These and other aspects and advantages of the present invention are described in the following detailed description of the invention.

### BRIEF DESCRIPTION OF THE DRAWINGS

FIG. 1A is a schematic illustration of the circulatory system with particular emphasis on the pathways of bodily microfluids.

FIG. 1B is a schematic illustration of the fluid dynamics in a body tissue.

FIG. 1C is a schematic illustration of the left venous angle and illustrates the thoracic duct draining into the venous circulation.

FIG. 2 illustrates a system of the present invention for conducting fluid management therapy.

FIG. 3 illustrates a system of the present invention for conducting fluid management therapy in a blood vessel.

FIG. 4 illustrates a method for initializing pressure sensor outside the body according to the present invention.

FIG. 5 illustrates a method for displaying pressure data from a sensor inside a body vessel according to the present invention.

FIG. 6 illustrates a method for measuring pressure from a sensor inside a body vessel according to the present invention.

FIG. 7 illustrates a first preferred embodiment of fluid management catheter system according to the present invention

FIG. 8 illustrates a second preferred embodiment of fluid management catheter system according to the present invention.

FIG. 9 shows tabular data of thermal sensitivities associated with individual pressure sensors.

FIG. 10 illustrates an interventional suite used in a fluid management therapy according to the present invention.

FIG. 11 illustrates a preferred embodiment of an apparatus for measuring pressure from a catheter fiber optic pressure sensor according to the present invention.

FIG. 12A illustrates a preferred embodiment of a piezoresistive pressure sensor used in a catheter according to the present invention.

FIG. 12B illustrates a preferred embodiment of a carrier platform for mounting the piezoresistive pressure sensor and securing inside a catheter according to the present invention.

FIG. 12C illustrates another preferred embodiment of a piezoresistive pressure sensor mounted on a carrier suitable for a catheter according to the present invention.

FIG. 13 illustrates a preferred embodiment of piezoresistive measurement circuit connected to a controller according to the present invention.

FIG. 14A illustrates a preferred embodiment of a motor control circuit according to the present invention.

FIG. 15 illustrates a circuit diagram of a safety system for a fluid management catheter according to the present invention.

FIG. 16A illustrates an unfiltered current signal containing noise generated by an operating motor.

FIG. 16B illustrates a preferred embodiment of a filter designed to remove unwanted noise generated by an operating motor from a current signal according to the present invention.

FIG. 16C illustrates a filtered current signal that may be used as an input into a safety system.

FIG. 17 shows a graph of current versus time in one embodiment of the motor control safety system.

FIG. 18 shows a graph of speed versus time in one embodiment of the motor control safety system.

FIG. 19 shows a schematic representation of hardware for one embodiment of a motor controller safety system suitable for incorporation into the fluid management systems of the present invention.

### DETAILED DESCRIPTION OF THE PREFERRED EMBODIMENTS

Specific embodiments of the present invention are now described in detail with reference to the figures, wherein the reference numbers indicate identical or functionally similar elements. The terms “distal” or “proximal” are used in the following description with respect to a position or direction relative to the treating physician. “Distal” or “distally” are a position distant from or in a direction away from the physician. “Proximal” or “proximally” or “proximate” are a position near or in a direction toward the physician.

The following detailed description is merely exemplary in nature and is not intended to limit the invention or the application and uses of the invention. In the present disclosure, the singular forms “a”, “an” and “the” includes the plural reference. It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination.

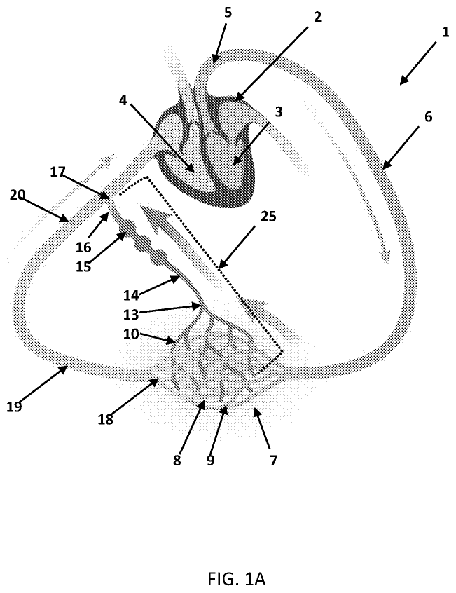

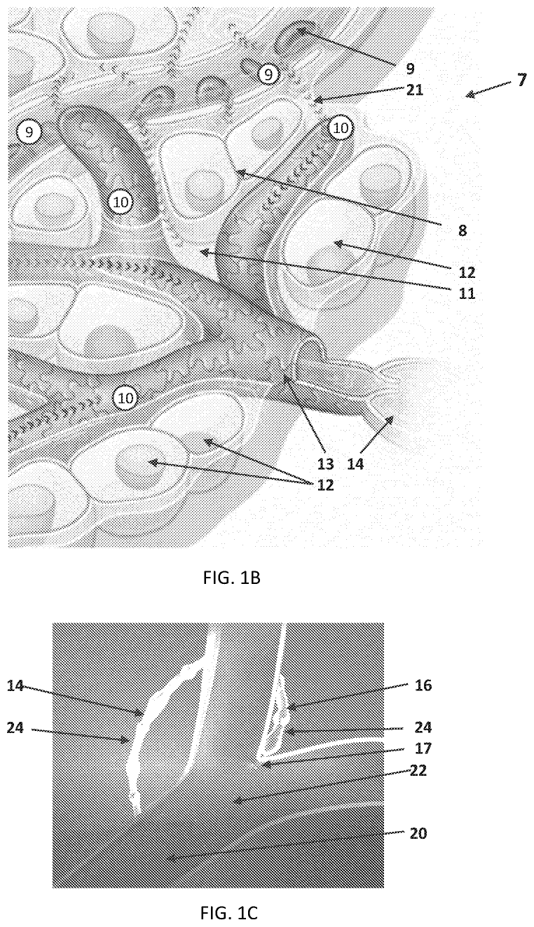

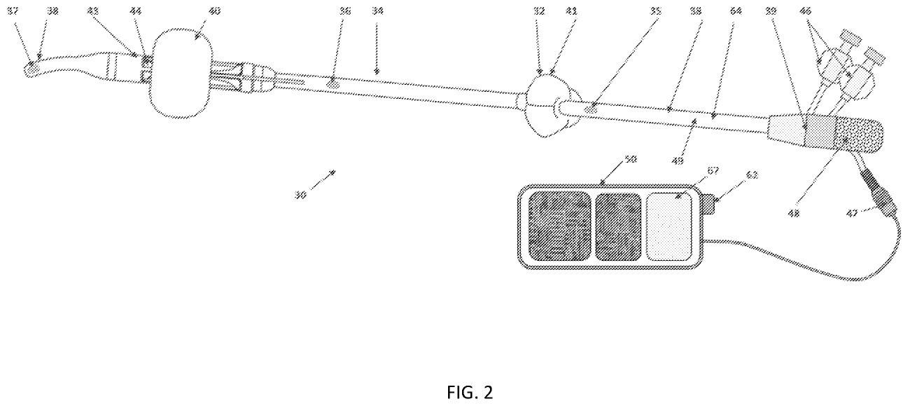

FIG. 1A shows schematic illustration of human microfluidic circulatory system (1). According to the present invention, the microfluids of the circulatory system (1) may be interpreted to include the following:
(a) the extracellular “watery” components of blood and some plasma proteins,
(b) extracellular interstitial fluids,
(c) lymph fluids, and
(d) chyle.

FIG. 1A illustrates a left heart pump 3 pumping oxygenated blood around the arterial system 6 through the aorta 5 in the first instance and progressively through branch vessels until the blood enters the bodily tissues 7. In the body tissues 7, the blood passes through tiny vessels (capillaries) 9 and in the capillaries is separated/filtered. Some of the low molecular weight components of the blood, the microfluids, can pass through the walls of the capillaries 9 and enter the interstitial tissue 8 while blood cells and larger blood molecules stay in the capillaries and move into venules 18 then to deep veins 19 and are returned to the right heart by the great veins 20.

The microfluids entering the interstitium 8 in the capillary bed 7 fill the spaces between the interstitial cells 12 referred as the interstitial space 11 and bathe the interstitial cells 12 in fluid that is riche in oxygen and nutrients. Blood separated microfluid entering the interstitial tissues 8 is referred as interstitial fluid. In the process of bathing the interstitial cells, the interstitial fluid carry oxygen and nutrients to these cells and toxins and waste products are allowed to move from the interstitial cells 12 into the interstitial fluids.

The interstitial fluid is collected by lymphatic capillaries 10 which are abundant in the interstitial tissues 8 and the fluid is conveyed by the lymphatic system 25 back to the venous compartment. Interstitial fluid entering the lymphatic capillaries 9 is referred as lymph fluid. In the lymphatic vessels of the gut, this fluid is referred as chyle due to the absorption of fat molecules into lymph fluid in the small intestines of the gut.

The lymphatic system 25 is a complex drainage network that consists of (i) lymphatic collecting vessels which receive fluid from the lymphatic capillaries 10 and carry it to large lymphatic vessels, (iii) lymphangions 14 which are miniature tubular pumping elements with one way value inlets and outlets, smooth muscle cells in its wall structure and a signaling system that controls its pumping action, (iv) lymph nodes 15 which are small multifunctional glands that filter lymph and have other immune functions, (v) lymphatic ducts 16 that collect lymph fluid from the upstream part of the lymphatic system 25 and connect with the great veins 20 via a lymph duct outlet located above the heart 4 to return the lymph to the blood circulatory system.

According to FIG. 1B, blood is carried to the tissue via the arterial capillaries 9 and some of the smaller (low molecular weight) components of blood cross the tine wall of the capillary 9 under hydrostatic pressure and enter the interstitial tissue 8. The chevron arrows 21 in the graphic highlight the flow pathway of microfluids first crossing the capillary fluids and then entering the lymphatic capillaries 10 as lymph fluid. The lymphatic capillaries 10 then meet with collecting lymphatic vessels 13 and farther downstream the lymph fluid passes through the valved system of lymphangions 14.

FIG. 1C shows a downstream region of the lymphatic system 25 where the large lymphatic vessels drain into the venous system at the great veins above the right heart 4. While anatomic variations are common, most people have two lymphatic duct vessels 16, the right lymphatic duct and the left thoracic duct 24. FIG. 1C shows the left thoracic duct 24 draining into the great veins 20 at the venous angle 22. The thoracic duct 24 (and the right lymphatic duct) has an outlet 17 and the outlet 17 is a valved structure that allows lymph fluid to enter the blood but prevent blood from entering the lymphatic system 25.

A lymph flow catheter system 30 according to the present invention is illustrated in FIG. 2 for treating a patient with a lymph flow deficit. The lymph flow deficit may be due to (i) an elevated central venous pressure, (ii) acute decompensated heart failure, (iii) lymphedema, (iv) edema, (v) ascites, (vi) heart failure, or (vii) kidney failure.

In a preferred embodiment of the lymph flow catheter system 30 according to the present invention, the lymph flow catheter system 30 includes a lymph flow catheter 34 and a console 50. The lymph flow catheter 34 includes a proximal restrictor 41 and a distal restrictor 40 with at least one the restrictors capable of defining a fluid flow path. Both the proximal restrictor 41 and distance restrictor 40 are depicted in the inflated state. In the preferred embodiment, the proximal restrictor 41 includes the fluid flow path. The catheter 34 includes a reduced pressure zone between the proximal and distal restrictors and the system 30 is configured to reduce the pressure in the reduced pressure zone and maintain the pressure in the reduced pressure zone over time. Since the proximal restrictor 41 includes a fluid flow path, a reduced volume of fluid flows across the proximal restrictor 41 and a reduced pressure zone between the proximal and distal restrictor can easily be maintained. For example, the fluid flow path reduces the volume of fluid entering the target zone and thereby making it easier to effect and sustain a pressure reduction in the target zone.

The lymph flow catheter 34 further comprises a blood pump 43 and blood pump 43 pumps blood from the reduced pressure zone to a region downstream of the reduced pressure zone. In one preferred embodiment of the blood pump 43 according to the present invention, the blood pump 43 includes an impeller pump with an impeller 44, an impeller housing 45, a motor 48 and a drive shaft 49 connecting the motor 48 to the impeller 44. In another embodiment, the console 50 wirelessly controls the operation of the motor 48 through communication with a local receiver element connected to the motor 48. The flow path is preferably configured such that a controlled flow of blood always enters the reduced pressure zone during the procedure. The controller of the console 50 controls the rotation of the impeller 44 to maintain the pressure in the reduced pressure zone at a constant level even if the pressures in upstream or downstream blood vessels change due to postural changes of the patient, or due to a reduction in venous volume due to diuresis or otherwise.

Preferably, the catheter 34 includes pressure sensors for monitoring pressure within a blood vessel. In one preferred embodiment of the catheter 34 according to the present invention, the catheter 34 includes three pressure sensors. A first pressure sensor 35 upstream of the proximal restrictor 41, a second pressure sensor 36 located between the first and second restrictors 40, 41, and a third pressure sensor 37 distal to the distal restrictor 40. The catheter 34 may include sensor lumens 38 in the wall of the catheter 34. The sensor lumens 38 extend proximally to facilitate intersection with one or more pressure sensor connectors 47. The pressure sensors 35,36, 37 may be configured for placement in sensor lumens 38. The pressure sensors 35, 36, 37 may include a sensing element and a sensor cable 64, the sensing element configured to sense the pressure and generate a signal and the sensor cable 64 configured to transmit the signal. The one or more sensing elements may be configured on the surface of the catheter 34. The one or more sensing elements may be configured in a lumen of the catheter 34. The one or more sensing elements may be configured at a proximal location and hydrodynamically connected to the vessel via a lumen through an opening in the wall of the catheter 34. The catheter 34 may include a sensor lumen outlet, the sensor lumen outlet including an opening or a skive in the wall of the catheter to hydrodynamically connect the sensor lumens 38 to bodily fluid adjacent to the sensor lumen outlet.

The pressure sensors 35, 36, 37 may include optical fiber (MOMS) pressure sensors. The pressure sensors 35, 36, 37 may include resistive or capacitive (MEMS) based pressure sensors. The catheter 34, pressure sensors 35, 36, 37 and controller 50 may be configured for monitoring pressure during the intravascular fluid management procedure. The catheter 34 may be operably connected to the controller 50. In a preferred embodiment according to the present invention, the operable connection includes connecting the pressure sensors 35, 36, 37 to the controller 50 for relaying sensed data for processing.

The system 30 may include stored thermal sensitivity data and/or drift characteristic data for the pressure sensors 35, 36, 37. The stored thermal sensitivity data and/or drift characteristic data may be stored on the controller 50 or a storage element of the catheter 34 or on a memory device connected to the system 30. Thermal sensitivity data and/or drift characteristic data allows the controller 50 to calculate an offset which can be applied when the temperature changes from a first temperature to a second temperature. The offset depends on the type of pressure sensor used and can even be specific to each pressure sensor. The offset ensures that the controller 50 is using accurate data on bodily fluid pressures notwithstanding a change in the operating temperature of the pressure sensors 35, 36, 37. Calculating an offset allows the controller 50 to measure pressure more accurately across a range of temperatures.

The controller 50 may include one or more ambient sensors 62 to measure ambient conditions including ambient pressure and ambient temperature. The one or more ambient sensors 62 are in contact with ambient conditions and include a data connection to the controller 50. The one or more ambient sensors 62 may be on the exterior surface of the controller 50 or may be integrated into a console on which the controller 50 is mounted. Alternatively, the one or more ambient sensor 62 may be wirelessly coupled to the controller 50. The one or more ambient sensors 62 provide accurate data to the controller 50 on ambient conditions. The controller 50 reads the thermal sensitivity data and/or drift characteristic data stored on the system 30, reads the ambient data provided by the ambient sensors 62, reads the pressure data provided by one or more of the pressure sensors 35, 36, 37 and calculates an offset for catheter operation in a body environment.

The controller 50 may also calculate pressure differences between two or more of the pressure sensors 35, 36, 37. The controller 50 provides the data of one or more of the pressure sensors 35,36, 37 to the treating physician. In one of the preferred embodiments, the pressure sensors 35, 36, 37 includes cables for communicating sensed data to the controller 50 and the cables extend from the sensors to the controller 50 for the transmission of sensed data. The system 30 is configured such that the at least one pressure sensors 35, 36, 37 are initialized in advance of conducting a fluid management procedure.

In another preferred embodiment of the system 30 according to the invention, the system 30 includes an intravascular catheter 34 with a first pressure sensor 35, a controller 50 with an ambient sensor 62 and a display 67. The first pressure sensor 35 (or 36 or 37) is mounted on the intravascular catheter 34 and is advanceable with the intravascular catheter 34 and is connected to the controller 50 and transmits pressure sensor data to the controller 50. The ambient sensor 62 is mounted relative to the controller 50 at a location where it can sense ambient pressure (without interference from local heat or cool sources). The ambient sensor 62 may be mounted on the controller 50 or in the controller 50 or on a console that carrier the controller 50 or spaced apart from the controller 50 but in all cases, the ambient sensor 62 is in communication with the controller 50 and transmits data on ambient conditions (pressure, temperature, humidity etc.) to the controller 50. In one preferred embodiment of the ambient sensor 62 according to the invention, the ambient sensor 62 is an absolute pressure sensor.

In second preferred embodiment, the ambient sensor 62 is a pressure sensor and a temperature sensor.

The system 30 includes thermal sensitivity data and/or drift characteristic data and calibration data stored on or connected to the controller 50. At the start of the procedure when the first pressure sensor 35 and the ambient sensor 62 are in a common environment the controller 50 is configured to read the pressure data of the first sensor 35 (or 36, or 37) and read the pressure data of the ambient sensor 62 and calculate an offset that is related to the difference between the reading from the first sensor 35 and the ambient sensor 62, the thermal sensitivity data and/or drift characteristic data of the first pressure sensor 35 and the calibration data of the first sensor 35.

In another variation, the offset is calculated based on the absolute reading of the first pressure sensor 35 (or 36 or 37), the absolute reading or the ambient sensor 62, the thermal sensitivity data of the first pressure sensor 35 (or 36 or 37) and/or drift characteristic data of the first pressure sensor 35 (or 36 or 37) and the calibration data of the first pressure sensor 35 (or 36 or 37).

The controller 50 may be configured to store the value of the offset for use during the procedure or the value may be stored on a memory device. The system 30 is further configured such that when the catheter 34 is inserted into a human body then the display 67 presents pressure data from the first pressure sensor 35 (or 36 or 37) modified by the application of the offset. The pressure data may be presented on the display 67 numerically, charted as a function of time, as a bar chart representation or in other forms that are standard for such data. The controller 50 may include a mobile cart 68 (stand).

The catheter 34 may be configured to increase or decrease bodily fluid pressure in at least a part of the vascular system. The catheter 34, in operation may be adjusted based on pressure measurements of the first pressure sensor 35 (or 36 or 37) and the offset. In one embodiment the first pressure sensor 35 (or 36 or 37) includes an absolute pressure sensor and the ambient sensor 62 includes an absolute pressure sensor.

In another preferred embodiment of the intravascular catheter 34 according to the present invention, the intravascular catheter 34 includes a passive state and an activated state. The passive state includes a delivery configuration, and the active state includes a treatment state.

In a preferred embodiment of the present invention, the initialized pressure reading includes a pressure reading that is adjusted for the ambient pressure in the local region (room, theatre, critical care unit or Cath lab) where the fluid management procedure is being conducted.

In another preferred embodiment of the present invention, the initialized pressure reading includes a pressure reading that is adjusted for the difference between the ambient temperature and the temperature of bodily fluids in the patient.

In yet another preferred embodiment of the present invention, the initialized pressure reading includes a pressure reading that is adjusted for the calibration data or drift characterization data of the of the first pressure sensor 35 (or 36 or 37).

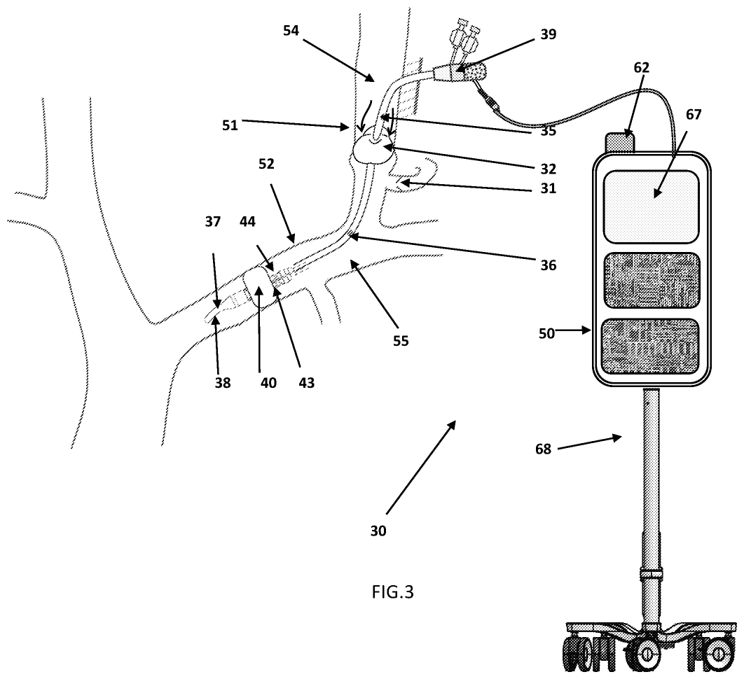

FIG. 3 shows the system 30 as illustrated in FIG. 2 when deployed inside a blood vessel. The intravascular catheter 34 is shown with its distal region, includes the catheter tip 38, the impeller pump 43 and the distal balloon 40 placed in one of the great veins and in this instance, the innominate vein 52. The catheter 34 extends proximally through the internal jugular vein 51 and then it crosses the internal jugular vein wall and extends exterior of the patient. At a proximal portion of the catheter 34 is a hub 39. The hub 39 may include any number of ports 46 (e.g., a flush port, restrictor inflation ports), a connector 47 for electronic communication with the pressure sensors 35, 36 and 37, and a motor 48. The motor 48 may be connected to an impeller pump 43 which is disposed at a distal portion of the catheter 34.

The impeller pump 43 may include an impeller 44 that is housed within an impeller housing in the distal portion of the catheter 34. The impeller housing may further include a number of inlets and outlets for allowing blood flow to move through the distal portion of the catheter 34. The distal balloon 40 is mounted on the impeller housing and is shown in the inflated state where it provides a seal against the wall of the innominate vein 52. In this configuration, the impeller 44 and impeller housing receive blood through the inlets to the impeller pump 43 and pump it out the outlets while the inflated distal balloon 40 prevents downstream blood from flowing retrograde. In this way the catheter 34 maintains a pressure gradient in the vessel that is in opposition to the pressure gradient in the vessel. Blood moves from the proximal side of the distal balloon 40 to the distal side of the distal balloon 40 even though the pressure is lower on the proximal side of the distal balloon 40. With this arrangement the catheter 34 creates a pressure gradient in a vessel and causes blood to flow against the created pressure gradient in the vessel.

In a preferred embodiment and as illustrated in FIG. 3, the catheter 34 is deployed inside a blood vessel and the catheter 34 extends from a patient's internal jugular vein 51 and terminates inside an innominate vein 52, such that a region between the distal restrictor 40 and the proximal restrictor 41 align with a thoracic duct 31 of the patient. During a treatment, inflation of the proximal restrictor 41 and distal restrictor 40 define a target region for establishing a low-pressure region 55 via operation of the impeller pump 43. The creation of this low-pressure zone 55 then facilitates the drainage of lymph fluid from the thoracic duct 31 and into the blood circulation.

In one of the preferred embodiments of the proximal restrictor 41 according to the present invention, the proximal restrictor 41 may include a precision restrictor 32. The precision restrictor 32 is configured to expand and oppose the vessel wall and it includes a fluid flow path that allows some fluid from the proximal internal jugular region 54 to flow across the precision restrictor 32 and thereby helps maintain the low-pressure zone 55 at a target pressure by reducing the volume flow rate of fluid entering the low-pressure zone 55.

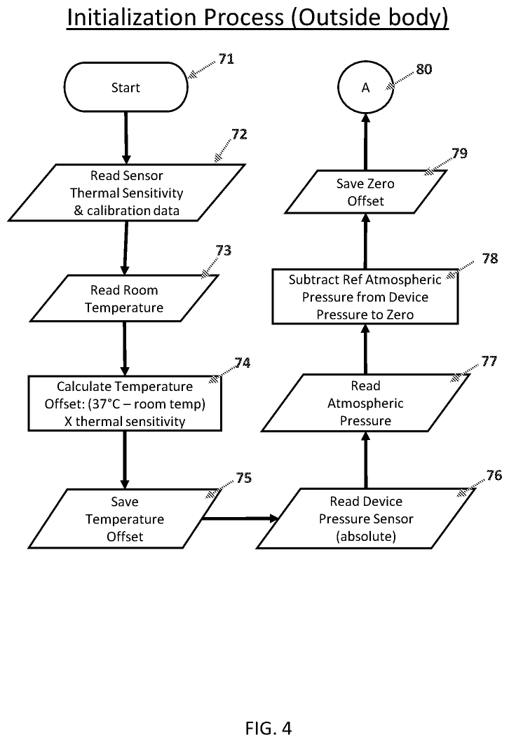

FIG. 4 is a flow chart illustrating the steps in initializing the sensors of the system 30 before inserting the catheter 34 into a patient. The initialization process is commenced when the catheter 34 is connected to the controller 50 but before the catheter 34 is inserted into the vein of the patient. The one or more ambient sensors 62 of the system 30 and the one or more pressure sensors 35, 36, 37 of the catheter 34 are in a common ambient environment for the measurements of the initialization process. The initialization process is ready to be started at step 71. Thermal sensitivity and/or drift characteristic data and calibration data of the pressure sensor 35 (or 36 or 37) are read by the console 50 in step 72. The room temperature data is read by the console 50 using the ambient sensor 62 at step 73. An offset is calculated at step 74. The offset calculation uses body temperature of the patient (normally 37° C.), the local ambient temperature of the room and the thermal sensitivity and/or drift characteristic data and calibration data of the pressure sensor 35 (or 36 or 37) to calculate the offset. The temperature offset is saved by the controller 50 at step 75. The controller 50 reads the pressure data of the pressure sensor 35 (or 36 or 37) at step 76. According to one preferred embodiment, the pressure sensor 35 (or 36 or 37) is an absolute pressure sensor. In second preferred embodiment, the pressure sensor 35 (or 36 or 37) is a relative pressure sensor.

The controller 50 reads the ambient atmospheric pressure data from the ambient sensor 62 at step 77. The controller 50 subtracts the atmospheric pressure data measurement from the data measurement of the pressure sensor 35 (or 36 or 37) at step 78 to calculate the offset required to zero the pressure sensor 35 (or 36 or 37) of the catheter 34 at step 79. The system zeros the pressure measurement displayed to the user on the display 67 at step 80 and the catheter 34 is now initialized and can be inserted into the patient.

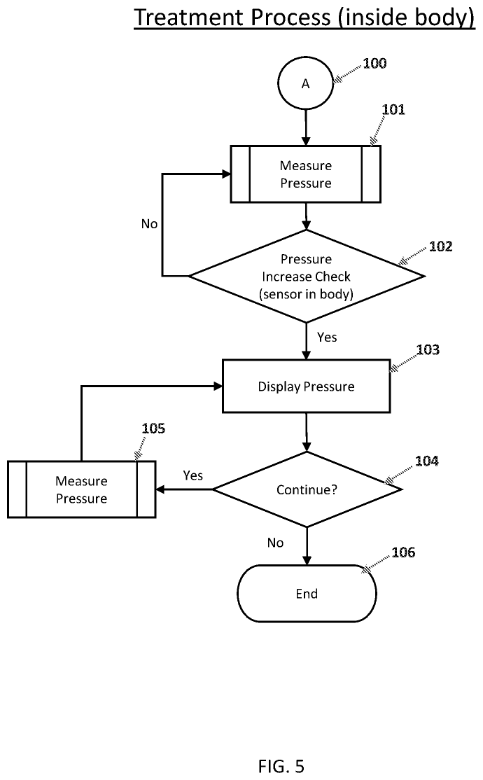

FIG. 5 is a flow chart illustrating the steps used by the system 30 to detect when the initialized catheter 34 is inserted in the body of the patient. The steps are important when therapy information is displayed to the treating physician on the display 67. It is important that the display 67 displays the correct data to the physician during the therapy and it is preferable that the system 30 displays the correct data to the physician for the stage of therapy without the need for user inputs. The display 67 may be a conventional screen or it may be a touch screen or other screens as are state of the art at the time.

At step 100, the catheter 34 has been initialized and a baseline pressure stored-the initialization pressure. At step 101, the controller 50 reads the pressure at the pressure sensor 35 (or 36 or 37) for a first pressure check. At step 102, the controller 50 checks if the value of the first pressure check has increased relative to the baseline pressure. If the first pressure measurement has not changed relative to the baseline pressure, then the controller 50 returns to step 101 and 102. When the pressure at the pressure sensor 35 (or 36 or 37) has increased above baseline then the controller 50 moves to step 103. In one of the preferred embodiments of the present invention, the step 103 may be skipped and a pressure displayed immediately. At step 103, the controller 50 displays the pressure reading of the pressure sensor 35 (or 36 or 37) on the display 67. This displayed pressure reading has been corrected for local atmospheric pressure and the temperature change from ambient and is an accurate measure of vascular pressure in the region of the pressure sensor 35 (or 36 or 37). The controller 50 checks if the user has continued the procedure at step 104. If yes, then at step 105, the controller 50 reads the pressure at the pressure sensor 35 (or 36 or 37) and at step 103, the controller 50 displays the pressure reading of the pressure sensor 35 (or 36 or 37) on the display 67. At step 104, the controller 50 again checks if the user has continued the procedure. If not the controller 50 proceeds to step 106 and terminates the procedure. It will be appreciated that the controller 50 may circle around the two loops ((i) 101/102 and (ii) 103/104/105) of FIG. 5 many times during therapy procedure.

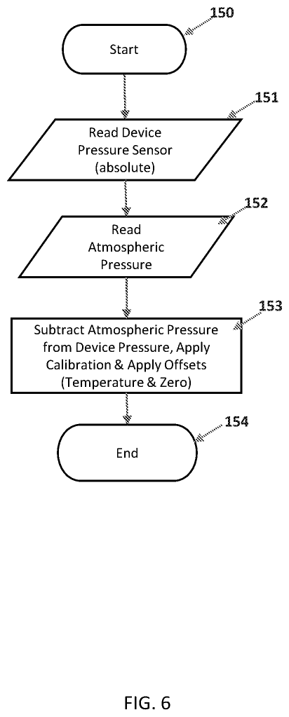

FIG. 6 shows steps used to determine a pressure during the of the invention. These steps involve correcting the pressure sensors 35 (or 36 or 37) of the catheter 34 for local atmospheric pressure. The steps of FIG. 6 are applicable to the systems 30 as illustrated in FIG. 2 and FIG. 3.

The steps are applicable to the system 30 when the catheter 34 of the system 30 is connected to the controller 50. The steps are initiated at step 150. At step 150 the operator initiates steps including steps 150-154. Alternatively, the connection of a hardware element of the system 30 to the controller 50 initiates these steps. At step 151, the controller 50 reads the data of the pressure sensor 35 (or 36 or 37). At step 152, the controller 50 reads the data of the ambient pressure sensor 62. At step 153, the controller 50 subtracts the atmospheric pressure measurement of the ambient pressure sensor 62 from the pressure reading of the pressure sensor 35 (or 36 or 37) and applies temperature sensitivity data and/or drift characteristic data and calibration data offsets to calculate accurately the pressure in the region of the pressure sensor 35 (or 36 or 37). Step 153 may include applying a mathematical calculation wherein the atmospheric pressure measurement and the pressure reading include variable parameters. Step 153 may include applying a mathematical calculation wherein the temperature sensitivity data and/or drift characteristic data and calibration data offsets comprise fixed parameters in the mathematical calculation. It will be appreciated that while the temperature sensitivity data and/or drift characteristic data and calibration data offsets may be fixed for a given temperature and so are fixed variables in one instant calculation they may vary from calculation to calculation. The thermal sensitivity data and/or drift characteristic data may also be variable with respect to time by a factor determined from the initial thermal sensitivity data and/or drift characteristic data. The controller 50 may display the pressure at the pressure sensor 35 (or 36 or 37) on the display 67. The steps end at step 154.

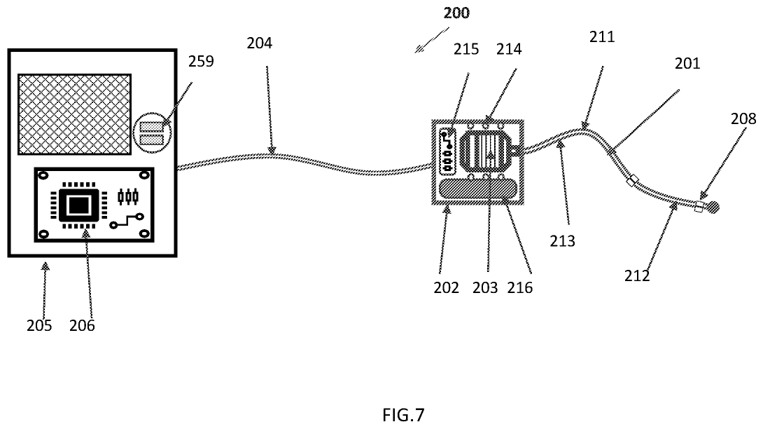

FIG. 7 shows another preferred embodiment of the fluid management system 200 of the present invention designed to treat patients suffering with edema, fluid congested ADHF patients, patients suffering ascites and patients with vascular congestion irrespective of the underlying etiology. The fluid management system 200 includes a controller 205, a hub 202, a fluid management catheter 201 and a connection apparatus 204. The controller 205 includes a controller processor 206, an ambient pressure sensor 259. The catheter 201 is configured for insertion into a circulatory system of the body of the patient and includes a catheter distal end 212 and a catheter proximal end 213, the distal end 212 of the catheter 201 extending into and through at least a portion of a vessel of a circulatory system and the proximal end 213 of the catheter 201 extending across the skin of the patient and exterior of the patient. The fluid management catheter 201 includes a first fluid sensor 208.

In one preferred embodiment of the first fluid sensor 208, the sensor 208 is configured to measure a fluid parameter, wherein the measured fluid parameter is influenced or controlled by the fluid management catheter 201.

In second preferred embodiment of the first fluid sensor 208, the sensor 208 is configured to measure a fluid parameter, wherein the measure fluid parameter is a measure of the progression of the therapy delivered by the fluid management system 200.

In third preferred embodiment of the first fluid sensor 208, the sensor 208 is configured to measure a fluid parameter, wherein the measured fluid parameter is used by the controller to control the operation of the fluid management catheter 201.

In fourth preferred embodiment of the first fluid sensor 208, the sensor 208 is a pressure sensor and the pressure sensor measures pressure in a circulatory system in the region of the fluid sensor 208.

In fifth preferred embodiment of the first fluid sensor 208, the sensor 208 is a flow sensor and the flow sensor measures the flow or the flow rate of fluid in a vessel of a circulatory system.

In sixth preferred embodiment of the fluid sensor 208, the sensor 208 is an impedance sensor and the impedance sensor takes an impedance measurement in the patient.

The catheter hub 202 further includes a motor 203, a noise and/or vibration damping arrangement 214, fluid sensor PCB 215 and a flushing manifold 216, the flushing manifold 216 including flush ports, stock cocks and/or other standard flushing and inflating components as are standard in interventional catheters. The motor 203 is configured to drive a fluid pump 256 in the catheter 201 and the speed of the motor 203 is controlled by the controller 205. The fluid sensor PCB 215 receives the signal from the fluid sensor 208 and processes the signal before feeding it to the controller 205 via the connection apparatus 204. The motor 203 is mounted in the hub 202 on a noise and/or vibration damping arrangement 214. The noise and/or vibration damping arrangement 214 is configured to allow the motor to operate at very high speeds without the patient or user experiencing excessive noise or vibration during the procedure.

In a preferred embodiment of the noise and/or vibration damping arrangement 214 according to the present invention, the arrangement 214 includes a plurality of rubber rings at least substantially encircling the motor 203 in the hub 202. The plurality of rubber rings may include split rings, or O rings. The plurality of rubber rings may include a rubber, synthetic rubber, elastomer, silicone or foam material. The plurality of rubber rings may include a vibration damping material. The plurality of rubber rings may include a sound absorbing or reflecting material.

In another preferred embodiment of the noise and/or vibration damping arrangement 214 according to the present invention, the arrangement 214 includes a plurality of strips, made from damping material, spaced apart and bonded to the motor 203 longitudinally.

In a preferred embodiment of the connection apparatus 204 according to the present invention, the apparatus 204 includes at least one cable connecting the catheter 201 and hub 202 to the controller 205. In the preferred embodiment, the hub 202 includes a hardware arrangement that allows a wireless connection between the hub and the controller 205. The hardware arrangement of the hub 202 includes a wireless transmitter and receiver, a microprocessor executing firmware and a battery. When operating wirelessly the fluid management system control parameters are received wirelessly from the controller 205. When operating wirelessly the hub 202 sends pressure parameters, pump parameters and information on battery state to the controller. In the preferred embodiment, the battery can be re-charged wirelessly and/or via a cable connection.

In another preferred embodiment, the battery can be removed by a user for charging and replaced while the fluid management system 200 continues to function via power from the connected cable 204 to the controller 205.

The ambient pressure sensor 259 is in communication with the controller 205 and provides the controller with real time measurement of atmospheric pressure in the room where the procedure is being carried out. The ambient pressure sensor 259 may be mounted on or near the controller 205 or it may be in another part of the room.

The fluid management catheter 201 may include a fluid flow restricting catheter, a fluid pumping catheter, a fluid aspiration catheter, a fluid infusing catheter, or a combination of these. The fluid management catheter 201 may include one or more lumens 211. The one or more lumens 211 are arranged in an efficient pattern to minimize the diameter of the catheter. In one pattern the lumens 211 of the catheter 201 may comprise a first central lumen substantially concentric with the OD of the catheter 201 and at least 2 other lumens in the annular wall of the catheter 201 and substantially equally angularly spaced apart.

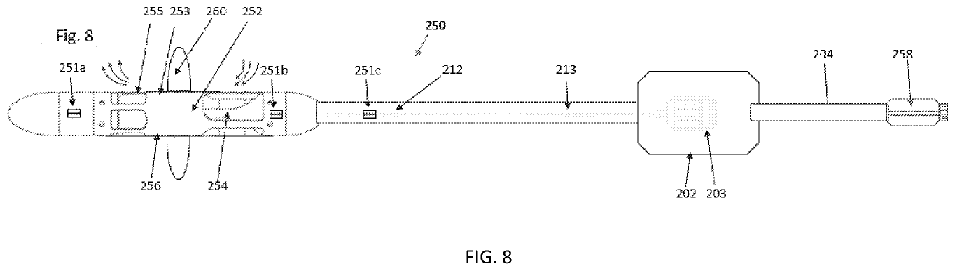

FIG. 8 illustrates one of the preferred embodiments of the fluid management catheter 250 of FIG. 7.

The fluid management catheter 250 comprises a catheter distal end 212 and a catheter proximal end 213, a pump assembly 256 at the distal end 212 and at the catheter proximal end 213 a hub 202, the hub 202 comprising a motor 203 and hardware, a connection cable 204 and controller connector 258 for connecting the fluid management catheter 250 to a controller 205. The pump assembly 256 includes an impeller housing 253, an impeller 252, the impeller 252 disposed inside the impeller housing 253 and the impeller 252 configured to rotate relative to the impeller housing 253. The pump assembly 256 further includes at least one inlet 254 through which fluid enters the pump assembly 256 under the influence of a negative pressure gradient created by the impeller 252, at least one outlet 255 through which fluid exits the pump assembly 256 and an expandable restrictor 260. The expandable restrictor 260 has a collapsed state and an expanded state and in the expanded state the expandable restrictor 260 apposes the wall of the vessel.

The fluid management catheter 250 of the system 200 further includes a plurality of fluid sensors 251. A first fluid sensor 251a is configured to sense pressure distal of the outlet 255 of the pump assembly 256. A second fluid sensor 251b is configured to sense pressure proximal of the inlet 254 of the pump assembly 256. A third fluid sensor 251c is configured to sense pressure in a proximal region of the vessel, closer to the region where the catheter shaft exits the vessel to the exterior of the body

In one preferred embodiment of the fluid management catheter 250 according to the present invention, the catheter 250 includes a fourth sensor configured to measure the pressure inside the expandable restrictor 260. The diameter of the expandable restrictor 260 may be controlled by pressure or by inflation volume. When the expandable restrictor 260 is in its expanded state and the pump assembly 256 is operating then a pressure gradient is maintained across the expandable restrictor 260 by the operation of the pump assembly 256. If the impeller 252 of the pump assembly 256 is operated at relatively higher speeds, then a greater pressure drop will be maintained across the expandable restrictor 260.

It will be appreciated that the methods for initializing pressure sensors, for monitoring pressure during an intravascular procedure, for providing safety protection hardware and for controlling impeller rotation as illustrated in FIG. 2, FIG. 3 and FIG. 4 apply to the system 200 of FIG. 7 and FIG. 8.

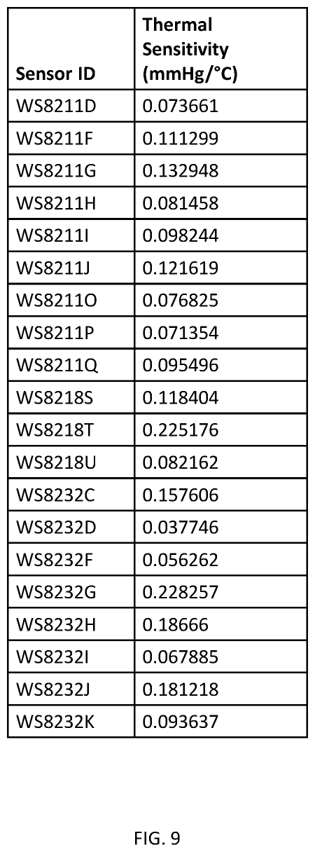

FIG. 9 shows a table detailing the thermal sensitivity data for a batch of sensors of the invention. It will be noted that each sensor in the batch has a slightly different sensitivity. For example, the sensor WS8211D has a thermal sensitivity of 0.073661mmHg/° C. while the sensor WS8211F has a thermal sensitivity of 0.111299. It will be noted that the second sensor has a thermal sensitivity that is 51% greater than the first sensor. The hardware and methods of the present invention compensate for these thermal sensitivities and for the differing thermal sensitivities and provide the user with accurate data irrespective which sensor is used during the fluid management procedure. Similarly, each sensor may have different drift characteristic data that is used by the hardware and methods of the present invention to provide accurate pressure data.

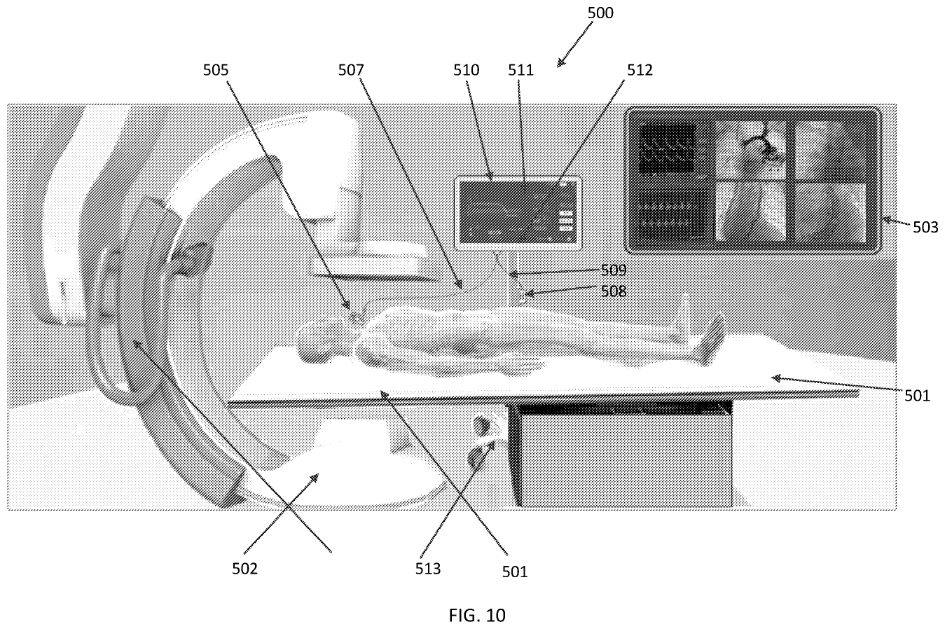

FIG. 10 shows a fluid management system of the present invention being used on a patient in a catheterization laboratory 500. The patient is represented on an operating table 501 and an imaging system 502 is used to visualize the placement of the fluid management catheter 505 during the procedure with images displayed on display screen 503. The fluid management catheter 505 is shown inserted into the internal jugular of the patient with the proximal end exterior of the patient. The catheter 505 includes at least one sensor and a sensor cable 507 extends from the catheter hub to the fluid management controller 512 of the console 510. The fluid management controller 512 includes a mobile cart (stand) 513, controller 512 and a display screen 511 for monitoring the procedure and for user touch screen inputs. The fluid management controller 512 further includes an ambient sensor (pressure and/or temperature) 508 and ambient sensor cable 509.

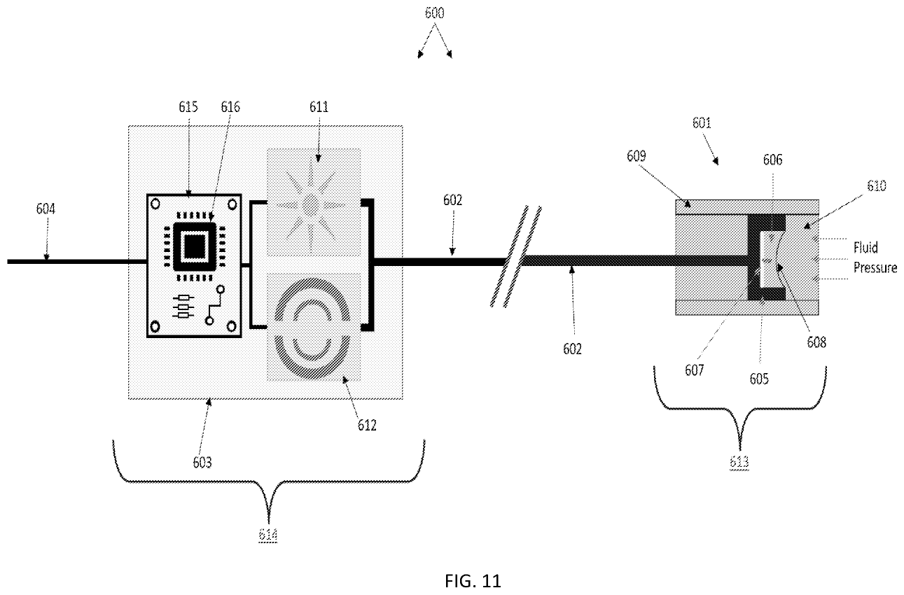

FIG. 11 shows a schematic representation of a pressure sensor apparatus 600 used in the system (30 or 200) of the present invention. The pressure sensor apparatus 600 includes a distal assembly 613 (shown in cross section) and a proximal assembly 614 (shown in schematic form). The distal assembly 613 is configured to be mounted on a catheter (34, 201 or 505) to sense fluid pressure in a vessel or cavity of a patient during a fluid management procedure.

In one preferred embodiment of the distal assembly 613 according to the present invention, the distal assembly 613 includes a pressure sensor 601. In one of the preferred embodiments of the present invention, the catheter (34 or 201) includes at least one lumen and the distal assembly 613 is configured for placement inside the at least one lumen, the at least one lumen further including an opening that puts the distal assembly 613 in fluid contact with fluid of the vessel or cavity. The distal assembly 613 further includes an optical fiber pressure measurement assembly comprising a flexible reflective membrane 608, a membrane housing 605 the membrane housing defining a cavity 606 into which the reflective membrane 608 can deflect, a second reflective membrane that provides a fixed reference 607 at the base of the cavity 606. The distal assembly further includes an outer protective housing 609, the outer protective housing 609 configured to protect the sensitive flexible reflective membrane 608 from damage and defining a pressure inlet 610. The protective housing 609 may include a rigid tubing made from metal or an engineering polymer.

The proximal assembly 614 includes an interferometer 603. The interferometer 603 is configured to send and receive light signals to the pressure sensor 601 through fiber optic cable 602. The interferometer 603 includes a light source 611, a detector 612, interferometer PCB 615 and an output cable 604 that relays the pressure measurement data in digital form to the controller (50, 205 or 512) of the system (30 or 200).

As fluid pressure increases in the vessel or cavity the pressure inlet 610 transmits fluid pressure to the flexible reflective membrane 608 and the flexible reflective membrane 608 deforms under the influence of the applied pressure. The position of the flexible reflective membrane 608 changes with respect to the fixed reference 607 and the connected interferometer 603 can sense this change as any distortion will result in a difference between the reflection of the fixed reference 607 and the reflection from the flexible membrane 608.

The interferometer measurement system 603 sends light signals to a connected pressure sensor 601 and the detector 612 detects reflected out of phase signals (interference) in the nanometer range from the connected fiber optic pressure sensor 601. The Interferometer 603 measures changes in the reflected optical signals to nanometer precision and uses stored calibration data from the individual sensor to convert the optical signals to a pressure value by performing a calculation on the processor 616 of the interferometer PCB 615. The interferometer 603 is integrated into a controller unit 50, 205 or 512 that transmits the digital pressure information to a display 67 or 511.

The light source 611 of the interferometer 603 is connected to and controlled by interferometer PCB 615 and is also connected to fiber optic cable 602 and is configured to generate and send light signals to the connected pressure sensor 601 via the fiber optic cable 602. The pressure sensing apparatus 600 is configured for incorporation into fluid management catheters (34, 201, or 505) of the invention. The pressure sensor 601 is configured to establish and maintain hydrostatic contact with fluid of the body vessel or cavity into which the catheter (34, 201 or 505) is placed. The pressure sensor 601 may be on the surface of the catheter (34, 201 or 505), extending from the catheter (34, 201 or 505) or in a hydrostatically connected lumen of the catheter (34, 201 or 505). If the pressure sensor 601 is in a hydrostatically connected lumen of the catheter (34, 201 or 505), then the lumen needs an opening sufficient to establish and maintain hydrostatic contact with fluids of the body cavity or vessel.

In one preferred embodiment of the present invention, the pressure sensor 601 is at a proximal end of a hydrostatically connected lumen. In second preferred embodiment of the present invention, the pressure sensor 601 is at a distal end of a hydrostatically connected lumen.

In one preferred embodiment of the present invention, the proximal assembly 614 is integrated into the catheter (34, 201 or 505). In second preferred embodiment of the present invention, the proximal assembly 614 is integrated into a distal segment of the catheter (34, 201 or 505). In third preferred embodiment of the present invention, the proximal assembly 614 is integrated into a proximal end of the catheter (34, 201 or 505) in another embodiment.

In one preferred embodiment of the present invention, the pressure sensor 601 and the interferometer 603 are in close proximity to one another or are integrated into a single assembly. Preferably the pressure sensor 601 is miniaturized for incorporation into a catheter (34, 201 or 505) or a catheter lumen.

In one preferred embodiment of the present invention, the catheter (34, 201 or 505) includes an indwelling catheter. With this embodiment indwelling means that at least a distal region of the catheter (34, 201 or 505) is configured for insertion into a body vessel or cavity for an extended duration, a period of hours, multiple days or longer or for permanent implantation. Where the interferometer 603 includes a part of an indwelling catheter (34, 201 or 505), the interferometer 603 includes a miniaturized assembly. It will of course be appreciated that a catheter (34, 201 or 505) or system (30 or 200) of the invention may incorporate multiple pressure sensing apparatus 600 to better deliver the fluid management therapy to the patient.

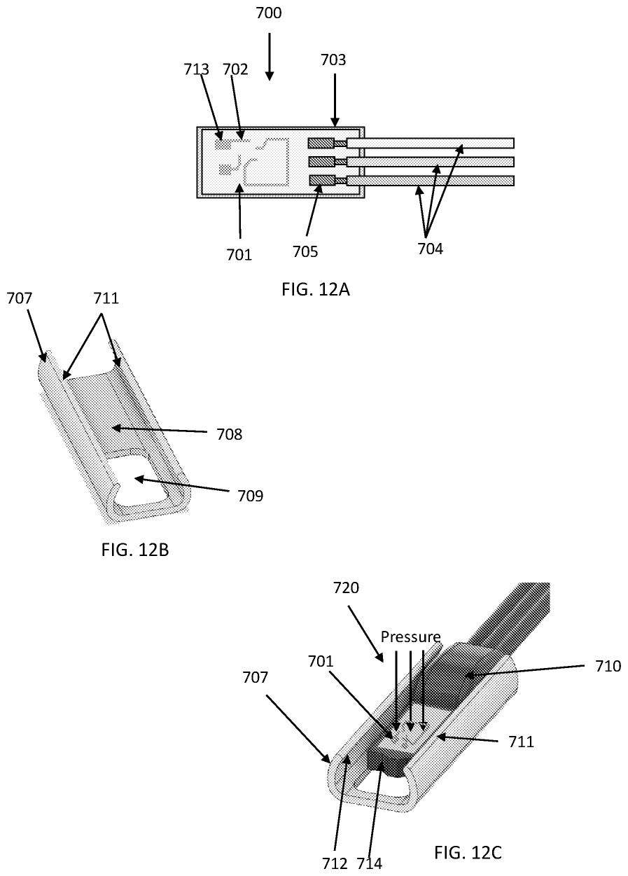

FIG. 12A, FIG. 12B and FIG. 12C illustrates a piezoresistive sensor 700 suitable for incorporation into the system (30 or 200) of the invention. FIG. 12A shows a top view of a piezoresistive sensor 700. The piezoresistive sensor 700 includes a piezoresistive silicon membrane 701, flexible circuit 702, sensor backing 703 onto which the flexible silicon membrane 701 and the piezoresistive circuit 702 are mounted, at least one sensor termination 705 and insulated sensor wiring 704. The flexible circuit 702 includes at least one piezoresistive resistor 713 and the flexible circuit 702 connects to at least one sensor wire termination 705. The at least one sensor wire termination 705 connects to the controller (50, 205 or 512) of the system (30 or 200).

FIG. 12B shows a 3D view of a sensor carrier 707. The sensor carrier 707 is geometrically shaped to facilitate the mounting of the pressure sensor 700. The sensor carrier 707 protects the pressure sensor 700 from a variety of mechanical forces and damage. In a preferred embodiment of the sensor carrier 707 according to the present invention, the sensor carrier 707 is configured to ensure that when the pressure sensor 700 is mounted on a catheter (34, 201 or 505) or in a catheter lumen that the piezoresistive silicon membrane 701 (and the piezoresistor 713) experience no stresses or forces other than the surrounding hydrostatic pressures.

In another preferred embodiment of the sensor carrier 707 according to the present invention, the sensor carrier 707 is configured to protect the piezoresistive silicon membrane 701 from forces of assembly or forces arising from the assembly or manufacturing process.

In yet another preferred embodiment of the sensor carrier 707 according to the present invention, the sensor carrier 707 is configured to protect the piezoresistive silicon membrane 701 from bending forces, torque forces, compressive forces, stretching forces experienced during the delivery or use of the catheter during the fluid management procedure.

In one preferred embodiment of the present invention, the sensor carrier 707 is configured to carry and protect the piezoresistive sensor 700. In this embodiment, the sensor carrier 707 includes a longitudinal channel 712 between two lateral walls 711. The longitudinal channel 712 is configured to facilitate the sliding of the pressure sensor 700 along the longitudinal channel 712 during assembly until it is completely within and protected by the sensor carrier 707. The longitudinal channel 712 may include a partially tube-like element or a profile geometry. The sensor carrier 707 may include a fixing arrangement 708 to hold the piezoresistive sensor 700 at a fixed location in the sensor carrier 707. The fixing arrangement 708 may be a snap fit or an adhesive bond or a formed element.

The piezoresistors of the silicon membrane 701 of the pressure sensor 700 change resistance as it experiences changes in pressure. The pressure sensor 700 further comprises a backing material 714 which provides mechanical support to the piezoresistive silicon membrane 701.

FIG. 12C shows 3D view of a preferred embodiment of the pressure sensor assembly 720 according to the present invention. The pressure sensor assembly 720 includes the pressure sensor 700 and the sensor carrier 707 in the assembled configuration. The pressure sensor assembly 720 in this state is ready for mounting on the catheter (34, 201 or 505) or for insertion into a lumen of the catheter (34, 201 or 505). The pressure sensor 700 is positioned in the longitudinal channel 712 of the sensor carrier 707 and the stress relief opening 709 ensures that the backing material 714 is suspended in the sensor carrier 707 and free from contact with carrier materials, bonding materials used in the assembly of the pressure sensor assembly 720 or bonding elements that include part of the pressure sensor assembly 720. The pressure sensor assembly 720 further includes a protective cover 710 that covers and insulates the sensor wire terminals 705. The protective cover 710 may be an adhesive covering, a reflowed polymer covering or a bonded component and, in all situations, the protective cover 710 is configured to prevent fluid ingress around the sensor wire terminations 705 and prevent short circuits between neighboring sensor wire terminations 705.

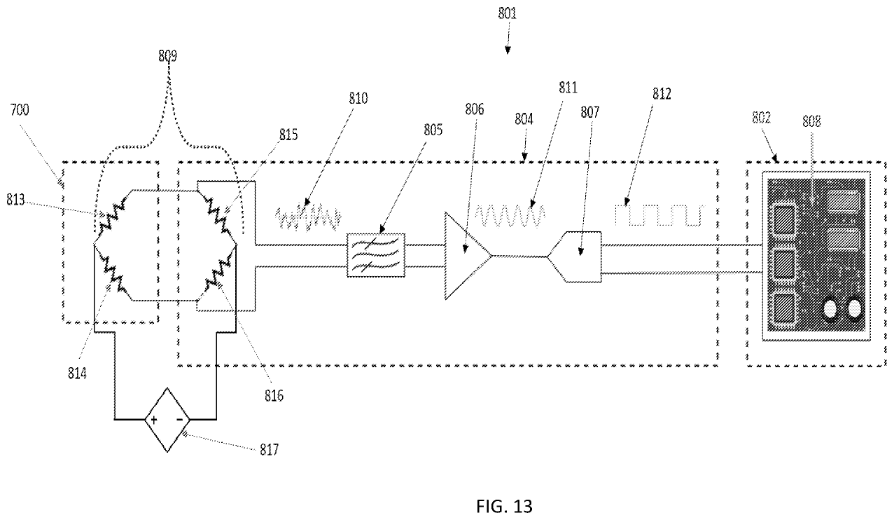

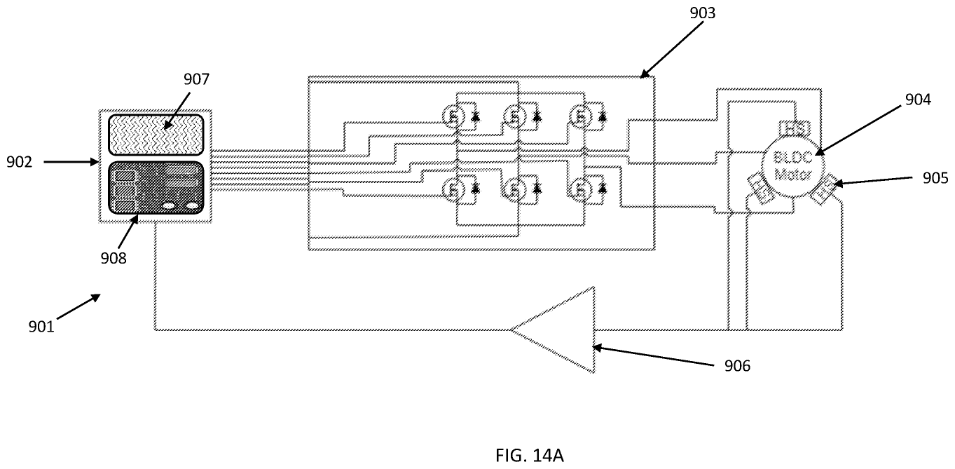

FIG. 13 shows a schematic representation of a piezoresistive sensor arrangement 801 according to the present invention. The piezoresistive sensor arrangement 801 is suited for incorporation into any of the fluid management systems (30 or 200) of the invention. The piezoresistive sensor arrangement 801 shows how the pressure sensor 700 as illustrated in FIG. 12A is built into a fluid management system (30 or 200).

In a preferred embodiment of the piezoresistive sensor arrangement 801 according to the present invention, the piezoresistive sensor arrangement 801 includes sensor controller 802, pressure sensor 700, and a signal processing assembly 804. The pressure sensor 700 includes a first piezoresistive resistor 813 and a second piezoresistive resistor 814 connected in series. The signal processing assembly 804 includes a first resistor 815 with a first known resistance, a second resistor 816 with a second known resistance, a filter 805, a buffer 806 and an analog to digital converter 807. The first resistor 815 and second resistor 816 are arranged with the first piezoresistive resistor 813 and a second piezoresistive resistor 814 to form a Wheatstone bridge 809. A constant voltage or current source 817 is used to supply energy to the bridge circuit. An unprocessed pressure signal 810 is generated by the response of the Wheatstone bridge 809 to a pressure and the unprocessed pressure signal 810 is filtered by passing the signal through filter 805 and buffer 806 to create a filtered analog pressure signal 811. The filtered analog pressure signal 811 is converted to a digital filtered pressure signal 812 by passing the filtered analog pressure signal 811 through an analog to digital converter 807. The digital filtered pressure signal 812 may then be used by the controller 802 in the management of the fluid management procedure. The controller 802 includes a processing unit 808 capable of measuring differences in voltage or current. The signal processing assembly 804 could be incorporated into the hub 202 with or without the sensor controller 802. The functions of controller 802 could be conducted by the controller (50 or 205 or 512) of the system (30 or 200) as described elsewhere in the patent.

In one of the preferred embodiments of the present invention, a constant current or constant voltage can be applied to the Wheatstone bridge 809 and the resulting change in voltages or current used to calculate a pressure by the controller 802 using sensor calibration data. Prior to sending the changes in current or voltage to the processor 808 on the controller 802 the signal is filtered, amplified, and converted to a digital form for transmission.

Referring to the FIG. 7, the pressure sensor 700 of the piezoresistive sensor arrangement 801 could be incorporated into a distal region 212 or a proximal region 213 of the catheter 201 or could be exterior of the patient in for example the hub 202 or the controller 205. The catheter 201 of the system may also incorporate a plurality of pressure sensors 700 and piezoresistive sensor arrangement 801.

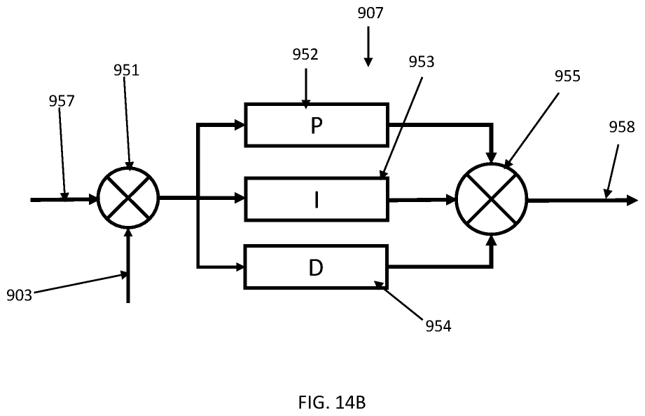

FIG. 14A and FIG. 14B show a motor control system 901 suited for incorporation into the fluid management system (30 or 200) of the present invention. It will be appreciated that in a fluid management procedure, particularly when of extended duration, that the fluid pressures parameters are changing due to a host of patient and system variables. These variables include (i) changes in blood volume, (ii) improvements in cardiac or other organ function during therapy, (iii) the use of restrictors, fluid pumps or other fluid management medical devices during therapy, (iv) positional changes by the patient, (v) the cardiac cycle, (vi) the respiration cycle, and (vii) adjunctive drug therapies used in the procedure. The motor control system 901 of the invention is configured to maintain the speed of the motor (48 or 203) notwithstanding changes in the fluid pressures parameters during therapy. While the motor control system 901 controls the speed of the motor (48 or 203), the controller (50, 205, or 512) of the fluid management system (30 or 200) determines from instant to instant the target speed for the motor (48 or 203).

In a preferred embodiment of the motor control system 901 according to the present invention, the motor control system 901 is configured to maintain the motor (48 or 203) that drives the pump 43 or the impeller 252 at the target speed. To maintain a target speed for the motor (48 or 203) during a therapy procedure, the motor controller 901 is configured to correct for errors introduced by process variables. The motor control system 901 includes a microcontroller or Field-Programmable Gate Array (FPGA) 902, transistor circuit 903, hall sensors 905 and signal conditioner 906. The microcontroller or FPGA 902 further includes a PID controller 907 which is configured to bring the motor 48 or 203 to the target speed in a controlled way and maintain that speed once achieved. In FIG. 14A, the motor is represented by numeral 904 but it will be appreciated that if the motor control system 901 is integrated into the fluid management systems (30 or 200), then the motor would be represented by the numeral 48 or 203. The hall sensors 905 measure the rotational speed of the BLDC motor 904 and the signal from the hall sensors 905 is passed through signal conditioner 906 and the refined signal is passed to the hardware 908 and PID Controller 907 of the microcontroller or FPGA 902. The microcontroller or FPGA 902 processes the refined signal and determines the energy delivery to the BLDC motor 904 and the transistor circuit 903 distributes the energy to the coils of the BLDC motor 904.

The microcontroller or FPGA 902 produces an output PWM (Pulse Wave Modulation) that is proportional to the speed calculated by the PID controller 907, that activate switches of the transistor circuits 902 which are connected to a power source. The PWM switching of the transistor circuit 903 allows electrical current to flow directly to the windings in the BLDC motor 904. To accurately control the motor 904, hall sensors 905 are included which send a signal on each revolution of the rotor of the BLDC motor 904. The hall sensor 905 feedback is conditioned by signal conditioner 906 for processing by microcontroller or FPGA 902.

In one of the preferred embodiments of the fluid management system (30 or 200) according to the present invention, the fluid management system (30 or 200) is configured for control of the motor 48 or 203 by current rather than by motor 48 or 203 rotor speed. With this embodiment a target pressure is achieved at the first pressure sensor 35, and/or the second pressure sensor 36 and/or the third pressure sensor 37 by monitoring and controlling the current delivered to the motor (48 or 203). The system (30 or 200) is as described in FIG. 2 or FIG. 7 includes a motor (48 or 203), at least one pressure sensor (35 or 36 or 37), PID controller 907, transistor circuits 902 and a controller (50 or 205 or 512). The controller (50 or 205 or 512) sends an output current to the motor (48 or 203) and monitors the pressure signal from the at least one pressure sensor (35 or 36 or 37) for at least a portion of a time interval. The PID controller 907 calculates a new motor current based on the pressure from the at least one pressure sensor (35 or 36 or 37) and the controller (50 or 205 or 512) produces an output PWM (Pulse Wave Modulation) to achieve the new current calculated by PID controller 907, that activate switches of the transistor circuits 902 which are connected to a power source. The motor (48 or 203) may be DC motor or a BLDC motor.

In one preferred embodiment of the present invention, the motor (48 or 203) is a BLDC motor or a DC motor without hall sensors and is configured with another means of measuring motor speed such an encoder, tachometer or a signal processor capable of detecting EMF feedback. The alternative speed measurement system may be used to produce an input to a control system. The motor (48 or 203) may also be controlled using compensation circuit (I×R) which increases the motor voltage with increasing motor current (increased torque).

FIG. 14B is a block diagram of the PID feedback loop of the PID Controller 907 of the motor controller system 901. The motor control system 901 is configured to keep the actual speed of the motor 48 or 203 that drives the pump close to the target speed for the motor 48 or 203. The motor control system 901 does this by calculating a correction to the actual motor speed using Proportional, Integral and Derivative terms. The difference between target motor speed and actual motor speed is provided as an input into the PID calculation. The calculated correction is the amount the speed the motor 48 or 203 is adjusted by. The PID terms are selected based on the expected response time of the indwelling pump 43 or 252 of the system 30 or 200. The PID feedback loop of the PID Controller 907 comprises error calculator 951, proportional term 952, integral term 953, derivative term 954 and adjustment calculator 955. The error calculator 951 receives actual speed values from the transit circuit 903 and target speed inputs 957 from the microcontroller or FPGA 902 or the system controller 50 or 205 or 512 and uses these inputs to calculate a correction. The PID feedback loop of the PID Controller 907 calculates an output speed 958 which is transmitted via transistor circuit 903 to the motor 904 or 48 or 203.

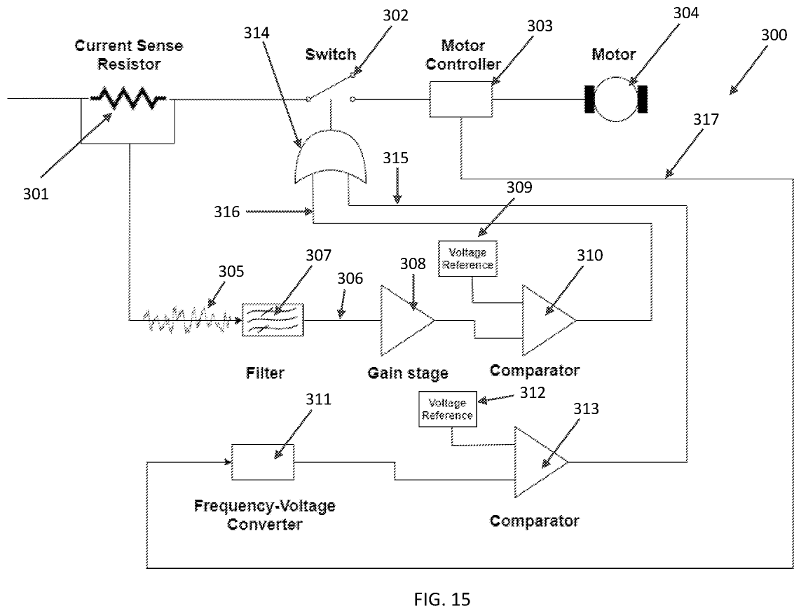

FIG. 15 shows a schematic representation of hardware for a motor controller safety system 300 suitable for incorporation into the fluid management systems (30 or 200) of the present invention. The hardware-based motor controller safety system 300 is configured to protect against a motor 48 or 203 of the system (30 or 200) operating above at least one safe limit due to a false input by either a user or from a software defect or otherwise. The motor controller safety system 300 includes a motor 304 (which is to be protected from over speed), a motor controller 303, switch 302, current sense resistor 301, filter 307, gain stage 308, first voltage reference 309, first comparator 310, frequency voltage converter 311, second voltage reference 312, and second comparator 313. When the switch 302 is in the closed position the motor 304 is driven by the motor controller 303. The switch 302 is connected to an OR gate 314 which deactivates the switch 302 if either input of the OR gate 314 is active. The first input of the OR gate 314 is the over speed signal 315. The second input of the OR gate 314 is the over current signal 316. If either signal is active OR gate 314 will produce an output that deactivates the switch 302. Deactivation of the switch 302 disables the operation of the motor controller 303 and the motor 304. The over speed signal 315 is activated when the voltage inputs to the comparator 313 are equal. The comparator 313 inputs are a reference voltage 312, which provides a fixed voltage safety limit, and the output of a frequency to voltage converter 311. The frequency to voltage converter 311 converts the actual motor speed 317 to a voltage that is proportional to the actual motor speed 317. The over current signal 316 is activated when the voltage inputs to the comparator 310 are equal. The comparator 310 inputs are a reference voltage 309, which provides a fixed voltage safety limit, and the output of the gain stage 308. The gain stage 308 amplifies a voltage, that is proportional to the current being demanded by the motor controller 303 and the motor 304, to usable level for voltage comparison. The gain stage 303 is connected to a filter which removes noise from the inbound current signal 305 generated by the motor controller 303 and the motor 304 operation. The filter is connected to the current sense resistor 301 which is in series the motor controller 303 when the switch 302 is closed. The voltage measured across the resistor serves as the input into the filter 307. In one embodiment the motor controller safety system 300 is configured to protect against an excessive current being transmitted to the motor 304. In one preferred embodiment, the motor controller safety system 300 is configured to protect against the motor 304 being operated at an excessive speed.

It will be appreciated that the motor controller safety systems 300 of FIG. 15 can be applied to any of the fluid management systems of the invention and the motor 304 could be the motors 48 or 203 as described elsewhere in this patent.

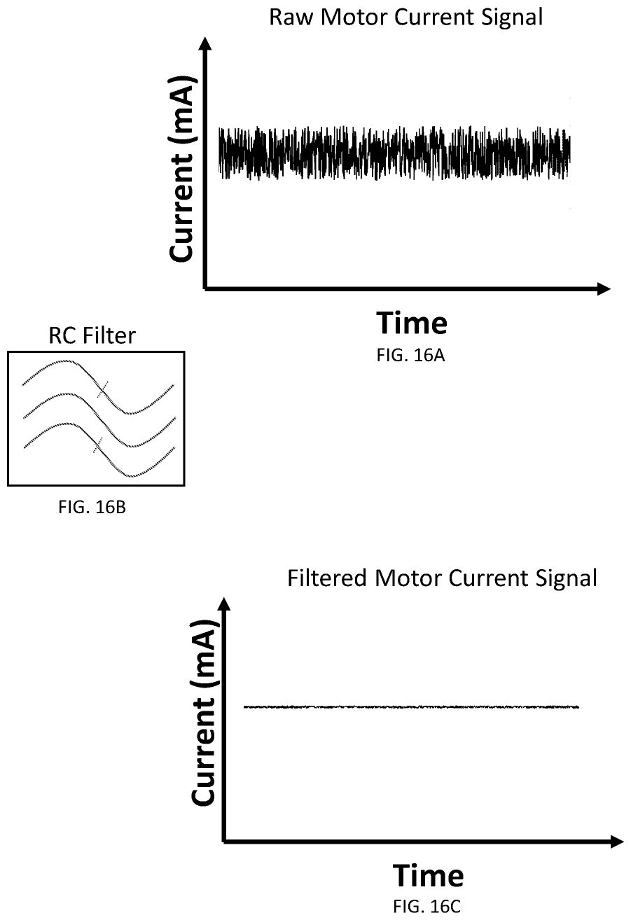

FIG. 16A shows a sample unfiltered signal 305 present in the circuit as illustrated in FIG. 15. The noise shown in the FIG. 16A is generated by high frequency switching action (PWM) of the motor controller circuit 303 and the motor 304 as illustrated in FIG. 14A and FIG. 14B, and FIG. 15. FIG. 16B shows an RC Filter element 307 implemented in hardware by means of a Resistor-Capacitor (RC) circuit. The RC circuit removes the high frequency noise and any unwanted low frequency noise. FIG. 16C shows the resulting signal after the noise has been removed. This ensures the over current detection element 302 of FIG. 15 can be activated at a precise threshold.

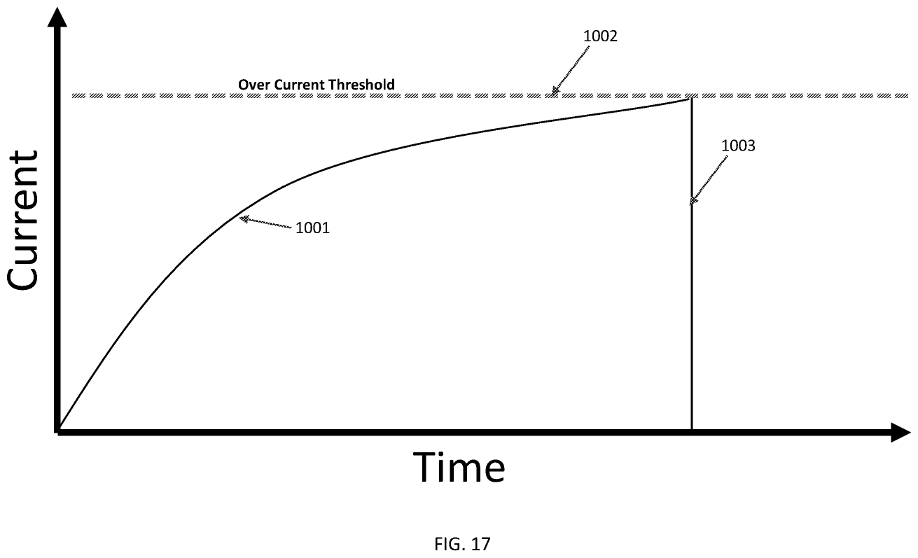

FIG. 17 shows a graph of current versus time in the motor control safety system 300 of FIG. 15. The graph shows the current 1001 rising gradually until it reaches the over current threshold 1002 of the motor control safety system 300. At the over current threshold 1002 the over current detection element switch 302 of FIG. 15 is activated and the motor controller 303 and motor 304 is switched off. In one preferred embodiment, the triggering of the current detection element 302 causes the current in the circuit to fall 1003 to zero. In second preferred embodiment, the triggering of the current detection element 302 causes the current in the circuit to fall to hardware default low value.

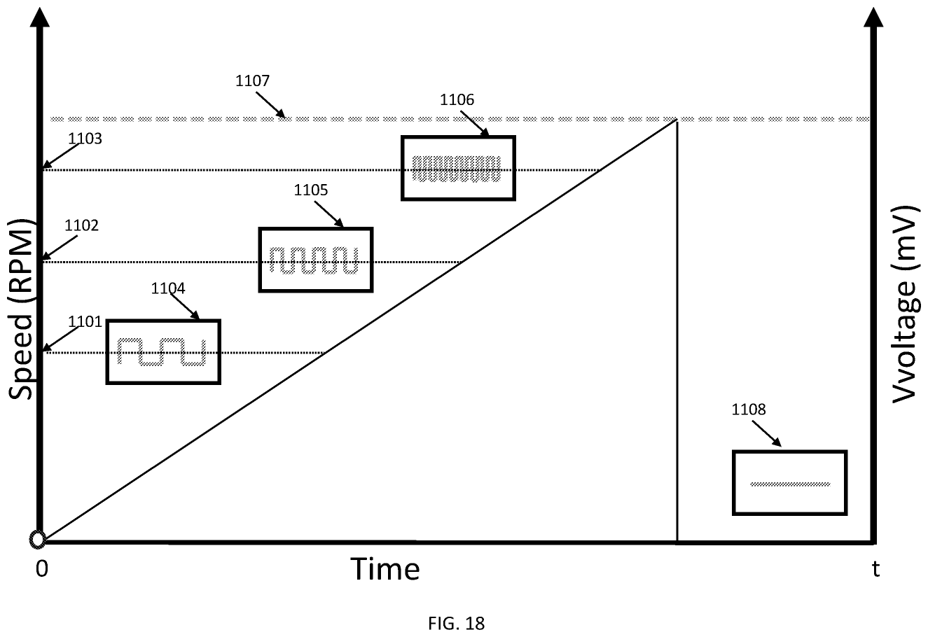

FIG. 18 shows a graph of speed versus time in the motor control safety system 300 as illustrated in FIG. 15. The graph shows the speed rising with respect to time. Output PWM signals, such as those generated by the controller 902 illustrated in

FIG. 14A. The chart contains a first speed 1101 speed signal 1104, a second speed 1102 speed signal 1105, a third speed 1103 speed signal 1106 and an over speed signal 1107. The PWM frequency increases relative to the required motor speed. The increase in frequency is converted to a voltage by the frequency to voltage converted 311 circuit in FIG. 15. At the over speed threshold 1107 the over speed detection element switch 302 of FIG. 15 is activated and the motor controller 303 and motor 304 is switched off. In one preferred embodiment, the triggering of the current detection element 302 causes the current in the circuit to fall to zero. In second preferred embodiment, the triggering of the speed detection element switch 302 causes the current in the circuit to fall to hardware default low current value 1108.

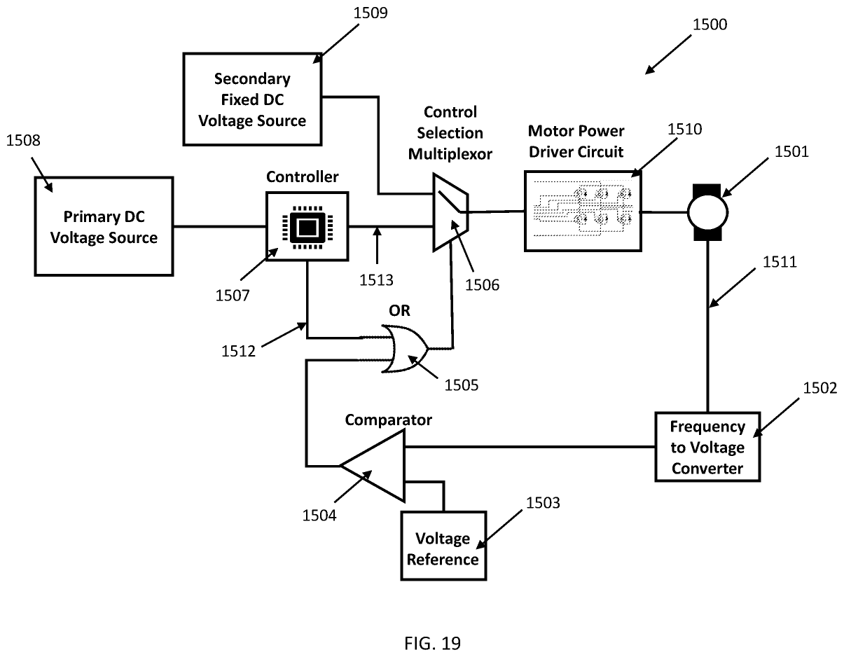

FIG. 19 shows a schematic representation of hardware for a motor controller safety system 1500 suitable for incorporation into the fluid management systems (30 or 200) of the present invention. The hardware-based motor controller safety system 1500 is configured to ensure the motor 48 or 203 of the system (30 or 200) continues to operate at a fixed low speed in the event the actual operating speed of the motor falls below a defined threshold or the firmware/software controlling the firmware ceases to operate.

The motor controller safety system 1500 includes a motor 1501 (which is to be protected from system failures by hardware), a frequency to voltage converter 1502, a reference voltage 1503, a comparator 1504, an OR gate 1505, a control selection multiplexor 1506, a motor controller 1507, a primary DC voltage source 1508, a secondary fixed DC voltage course 1509 and a motor power driver circuit 1510.

The Frequency to Voltage Converter 1502 converts the actual motor speed 1511 to a voltage that is proportional to the actual motor speed. The comparator 1504 compares the converted voltage to a voltage reference 1503. The voltage reference 1503 is a defined threshold voltage that the motor should not normally run at. It is a voltage that signifies that the motor controller 1507 or some other system failure has occurred. The comparator 1504 produces an output signal when the motor speed 1511 has fallen below the threshold. The output of the comparator 1504 is connected to the OR gate 1505. Also connected to the OR gate is an output from the motor controller 1507. The motor control has an output 1512 that is only activated in the event of a failure of the motor controller 1507. If either the comparator 1504 output or the motor controller 1507 output 1512 are active then the OR gate 1505 produces an active output. The output of the OR gate is connected to the control selection multiplexor 1506. An active signal from the OR gate 1505 causes the multiplexor 1506 to switch its input. The multiplexor 1506 has two inputs. The first input of the multiplexor is the motor controller 1507. The default state of the multiplexor 1506 is such that the motor controller 1507 is directly connected to the motor power driver circuit 1510. The motor controller is capable of producing an output 1513 that drives the motor power driver circuit. In one embodiment this may be multiple phased PWM signals. The motor controller 1507 is capable of varying the speed of the motor 1501 by varying the output 1513. The motor controller 1507 is capable of maintaining a target speed received from another circuit. In another embodiment it is capable of maintaining a target torque/current received from another circuit. The motor controller is connected to a primary DC voltage source 1508 that is separate from the secondary fixed DC voltage source 1509. The second input of the multiplexor 1506 is connected to the secondary fixed DC voltage source 1509. The secondary fixed voltage source 1509 is capable of producing a voltage at a defined threshold to support running the motor 1501 at a low speed. In one embodiment these may be multiple phased PWM signals. If the multiplexor is activated by a signal from the OR gate 1505 it will switch inputs to the secondary fixed DC voltage source 1509 resulting in the secondary fixed DC voltage source 1509 being connected directly to the motor power driver circuit 1510. The motor power driver circuit 1510 is connected directly to the motor 1501. In one embodiment the motor power driver circuit 1510 deliver the desired voltage and current to each coil of a BLDC motor. In another embodiment it drives a single phase DC motor.

In another embodiment the hardware system 1500 may be modified to include in addition a low current/torque detection circuit, using elements similar to elements of the system described in the hardware system 300, with an appropriate resistor value for the current sense resistor 301, to detect a low current that is compared by comparing two voltages, one being a low voltage, by means of a comparator that is connected to the OR gate 1505. In another embodiment the low current/torque detection circuit may substitute the low speed detection element.

In another embodiment the hardware system 1500 is combined with the hardware system 300 to provide a system that has safety elements including motor operation at a fixed low speed or current/torque in the event of the primary motor control system failure, disabling of the motor controller when current/torque limits are exceeded and disabling of the motor controller when a high speed limit is exceeded.

While there have been described what are believed to be the preferred embodiments of the present invention, those skilled in the art will recognize that other and further changes and modifications may be made thereto without departing from the spirit of the invention, and it is intended to claim all such changes and modifications as fall within the true scope of the invention.

What is claimed is:

1. A lymph flow catheter system for treating a patient with a lymph flow deficit, the system comprising
(a) a console, and
(b) a lymph flow catheter, wherein the lymph flow catheter comprises
(i) a pressure sensor,
(ii) a proximal restrictor,
(iii) a distal restrictor,
(iv) a reduced pressure zone between the proximal restrictor and the distal restrictor, and
(v) a blood pump, wherein the blood pump comprises an impeller pump which comprises
a) an impeller,
b) an impeller housing,
c) a motor, and
d) a drive shaft connecting the motor to the impeller, wherein the motor is controlled by the console.

2. The system of claim 1, wherein the lymph flow catheter comprises three pressure sensors.

3. The system of claim 2, wherein the lymph flow catheter comprises a first pressure sensor positioned upstream of the proximal restrictor.

4. The system of claim 2, wherein the lymph flow catheter comprises a second pressure sensor positioned between the proximal and distal restrictors.

5. The system of claim 2, wherein the lymph flow catheter comprises a third pressure sensor positioned distal to the distal restrictor.

6. The system of claim 1, wherein the lymph flow catheter further comprises one or more sensor lumen in the wall of the lymph flow catheter.

7. The system of claim 2, wherein the pressure sensors are configured in the sensor lumen.

8. The system of claim 1, wherein the pressure sensor comprises a sensing element and a sensor cable.

9. The system of claim 1, wherein the pressure sensor is selected from the group comprising of fiber optical MOMS pressure sensor and resistive or capacitive MEMS pressure sensor.

10. The system of claim 1, wherein the rotation of the impeller is controlled by the console to maintain the pressure in the reduced pressure zone.

11. The system of claim 1, wherein the console is attached to one or more ambient sensors.

12. A fluid management system for treating fluid management disorders, the system comprising a display, a controller with an attached ambient sensor, and an intravascular catheter with a mounted pressure sensor, wherein the intravascular catheter is connected to the controller.

13. The system of claim 12, wherein the ambient sensor is a pressure sensor and a temperature sensor.

14. The system of claim 12, wherein data received from the ambient sensor and the pressure sensor is presented on the display.

15. The system of claim 12, where the intravascular catheter is designed to a hub at the proximal end.

16. The system of claim 15, wherein the hub comprises a one or more ports, a connector, and a motor.

17. The system of claim 16, wherein the motor is connected to an impeller pump positioned at a distal end of the intravascular catheter.

18. The system of claim 17, wherein the impeller pump comprises an impeller housed within an impeller housing.

19. The system of claim 12, wherein the intravascular catheter comprises a distal restrictor and a proximal restrictor.

20. A method of treating a patient with fluid management disorder, the method comprising steps of
(a) providing a fluid management system comprising an intravascular catheter and a controller, wherein the intravascular catheter is mounted with a first pressure sensor and the controller is coupled with a second pressure sensor,
(b) providing thermal sensitivity and/or drift characteristic data and calibration data from the first pressure sensor,
(c) connecting the first pressure sensor to the controller before deploying the catheter inside the patient body,
(d) reading the first pressure sensor for a first pressure data in an ambient state,
(e) reading the second pressure sensor for a second pressure data in the ambient state,
(f) inserting the intravascular catheter into a blood vessel of the patient and reading a third pressure data with the first pressure sensor,
(g) calculating an offset of the intravascular catheter based on the difference between readings of step (d) and step (e), the thermal sensitivity data and/or drift characteristic data of the first pressure sensor and the calibration data of the first pressure sensor,
(h) subtracting the offset from step (g) from the reading of step (f) to derive a measurement of actual pressure in the blood vessel, and
(i) transmitting energy through the intravascular catheter based on the derived pressure measurement in the blood vessel.

21. The method of claim 20, wherein the intravascular catheter is an operable catheter.

22. The method of claim 21, wherein the operable catheter is configured to increase or decrease bodily fluid pressure or flow of the blood vessel.

23. The method of claim 21, wherein the operable catheter is adjusted based on pressure data of the first pressure sensor and the offset.

24. The method of claim 20, wherein the first pressure sensor is an absolute pressure sensor.

25. The method of claim 20, wherein the second pressure sensor is an absolute pressure sensor.

26. The method of claim 20, wherein the intravascular catheter can be configured in a passive state and an activated state.

27. The method of claim 26, wherein the intravascular catheter is deployed in the blood vessel in the passive state.

28. The method of claim 26, wherein the intravascular catheter is in the activated state once deployed in the blood vessel.

29. The method of claim 20, wherein the derived pressure measurement is a pressure reading adjusted for the ambient pressure in the region of the fluid management procedure.

30. The method of claim 20, wherein the derived pressure measurement is a pressure reading adjusted for the difference between the ambient temperature and the temperate of bodily fluids in the patient.

31. The method of claim 20, wherein the derived pressure measurement is a pressure reading adjusted for the calibration data of the first pressure sensor.

32. The method of claim 20, wherein the method further comprises step of displaying the derived pressure on a display screen.

33. The method of claim 21, wherein the operable catheter is configured to increase or decrease bodily fluid pressure or flow in one or more cardiac chambers.

34. The method of claim 20, wherein the fluid management disorder is acute decompensated heart failure, chronic heart failure, ascites, lymphedema, chronic kidney disease, cardiac insufficiency, cardiac value regurgitation, or plural effusions.

35. A method of monitoring pressure during an intravascular fluid management procedure, the method comprising the steps of
(a) providing a fluid management system comprising an intravascular catheter and a controller, wherein the intravascular catheter is operably connected to the controller and comprises a sensor,
(b) providing thermal sensitivity and/or drift characteristic data and calibration data from the sensor,
(c) reading the thermal sensitivity and/or drift characteristic data and calibration date for the sensor,
(d) reading ambient parameters in the procedure room,
(e) calculating an offset for the intravascular catheter in a patient body, and
(f) transmitting energy through the intravascular catheter based on the sensor data.

36. The method of claim 35, wherein the intravascular catheter is deployed into a blood vessel of the patient.

37. The method of claim 36, wherein the intravascular catheter extends from the patient's internal jugular vein and terminates inside an innominate vein.

38. The method of claim 35, wherein the intravascular catheter comprises a distal restrictor and a proximal restrictor.

39. A method of restricting fluid flow in a blood vessel, the method comprising the steps of
(a) providing a fluid management system comprising an intravascular catheter, a controller, and a display, wherein the intravascular catheter comprises a pressure sensor, a mounted restrictor, wherein the controller is connected to the pressure sensor and the display,
(b) deploying the catheter into the blood vessel and measuring the pressure data generated by the pressure sensor,
(c) filtering the measured pressure data at a selected permeability,
(d) displaying the filtered data on the display,
(e) expanding the mounted restrictor while displaying the filtered data, and
(f) detecting the point where the mounted restrictor contacts the vessel wall from the trend line of the filtered data.

40. The method of claim 39, wherein the step (d) display both filtered data and unfiltered data on the display.

41. The method of claim 39, wherein detecting step (f) comprises incrementally increasing the volume of the mounted restrictor while observing an absence of a corresponding change in fluid pressure.

42. The method of claim 39, wherein detecting step (f) comprises incrementally increasing the volume of the mounted restrictor while observing a substantially flat line on the displayed filtered data.

43. The method of claim 39, wherein expanding step (e) comprises expanding the mounted restrictor in a plurality of pressure increments.

44. The method of claim 39, wherein expanding step (e) comprises expanding the mounted restrictor in a plurality of volumetric increments.

45. The method of claim 39 further comprises the step of over expanding the mounted restrictor.

46. The method of claim 39, wherein the mounted restrictor comprises a diameter volume curve.

47. The method of claim 46, wherein the diameter volume curve detects the point when the mounted restrictor contacts the vessel wall.

48. The method of claim 39, wherein the mounted restrictor comprises a complaint and resilient restrictor.

49. A motor control system for pumping body fluids during an intravascular fluid management procedure, the system comprises
(a) a pump assembly configured for deployment inside a patient,
(b) a motor configured to drive a pumping element of the pump assembly,
(c) a hardware controller comprising a microprocessor and a software, wherein the software is configured with the hardware controller to operate the pump assembly,
wherein the hardware controller comprises a first circuit, a second circuit, a motor operation detection element, and a switch element,
wherein the motor operation detection element is configured to generate an electrical output proportional to an operative parameter of the motor,
wherein the switch element is configured to switch the operation of the motor from the first circuit to the second circuit when the operative parameter exceeds a predefined limit.

50. The system of claim 49, wherein the pump assembly comprise a blood pump.

51. The system of claim 49, wherein the pump assembly comprise a housing encasing the pumping element.

52. The system of claim 51, wherein the pumping element comprises an impeller.

53. The system of claim 49, wherein the switch element comprises a plurality of switch states.

54. The system of claim 53, wherein the plurality of switch states comprises a first state and a second state.

55. The system of claim 54, wherein during the first state, electron flow between the first circuit and motor is facilitated.

56. The system of claim 54, wherein during the first state, electron flow between the second circuit and the motor is blocked.

57. The system of claim 54, wherein during the second state, electron flow between the second circuit and motor is facilitated.

58. The system of claim 54, wherein during the first state, electron flow between the first circuit and the motor is blocked.

59. The system of claim 54, wherein the plurality of switch states further comprises a third state.

60. The system of claims 53 and 54, wherein switching the switch element from the first state to the second state causes the motor to operate at a preset rotation speed limit.

61. The system of claims 53 and 54, wherein switching the switch element from the first state to the second state causes the motor to operate at a preset upper rotation speed limit.

62. The system of claims 53 and 54, wherein switching the switch element from the first state to the second state causes the motor to operate at a preset standby rotation speed limit.

63. The system of claim 49, wherein the hardware controller further comprises a current detection element configured to generate an electrical output proportional to the current detection by the current detection element.

64. The system of claim 49, wherein the software provides a computational operation on the electrical output of the current detection element.

65. The system of claim 64, wherein the computational operation produces a computational output, which is used by the software to manage safe operation of the pump.

66. The system of claim 65, wherein the manage safe operation of the pump includes
(i) displaying a warning to the physician,
(ii) reducing or increasing the speed of the motor,
(iii) flushing a component of the pump assembly, and
(iv) terminating the treatment procedure.

67. The system of claim 49, wherein the hardware controller further comprises a motor torque detection element configured to generate an electrical output proportional to the torque detected by the torque detection element.

68. The system of claim 67, wherein the software provides a computational operation on the electrical output of the motor torque detection element.

69. The system of claim 68, wherein the computational operation produces a computational output, which is used by the software to manage safe operation of the pump.

70. The system of claim 69, wherein the manage safe operation of the pump includes
(i) displaying a warning to the physician,
(ii) reducing or increasing the speed of the motor,
(iii) flushing a component of the pump assembly, and
(iv) terminating the treatment procedure.

71. A fluid management system for treating fluid management disorders, the system comprising:
a controller having data and instructions stored therein;
a display operably coupled to the controller; and
a hub communicatively coupled to the controller, the hub comprising at least one connection point for connecting to an intravascular catheter,
wherein the controller is operable to receive data for pressure within a blood vessel of a patient and execute the instructions using the received data to control at least one device on the intravascular catheter to relieve a fluid management disorder.

72. The system of claim 71, further comprising an ambient sensor operably attached to the controller.

73. The system of claim 72, wherein the ambient sensor includes a pressure sensor and/or a temperature sensor.

74. The system of claim 72, wherein data received from the ambient sensor is presented on the display.

75. The system of claim 71, wherein the fluid management disorder is one selected from the group consisting of acute decompensated heart failure, chronic heart failure, ascites, lymphedema, chronic kidney disease, cardiac insufficiency, cardiac value regurgitation, and plural effusions.

76. The system of claim 71, wherein the hub comprises a one or more ports, a connector, and a motor.

77. The system of claim 76, wherein the motor comprising a connection connectable to a drive shaft of an impeller pump positioned at a distal end of the intravascular catheter.

78. The system of claim 71, wherein the instructions in the controller comprise computer program instructions operable to cause the controller to activate a restrictor and/or an impeller on the catheter.

79. The system of claim 71, wherein the system includes thermal sensitivity data and/or drift characteristic data and calibration data accessible to the controller.

80. The system of claim 79, wherein the system is operable to:
read intravascular pressure from a sensor and ambient pressure data of from a connected ambient sensor; and
calculate an offset using the intravascular pressure, the ambient pressure, the thermal sensitivity data, and/or the calibration data.

81. The system of claim 80, further wherein the system is operable to:
subtract the offset from the intravascular pressure to derive a measurement of actual pressure in the vessel.

82. The system of claim 81, further wherein the system uses the actual pressure to calculate and display a risk or degree of heart failure.

83. The system of claim 79, wherein the system uses the thermal sensitivity data and/or drift characteristic data and one or more temperature measurements to calculate an offset, wherein the offset is used to provide the controller with accurate data on bodily fluid pressures.

84. The system of claim 71, further comprising one or more sensors used to collect offset data, wherein the instructions are operable to use the offset data to calculate an offset, wherein the system uses the calculated offset to correct a measured bodily fluid pressure and provide a corrected bodily fluid pressure.

85. The system of claim 84, wherein the controller uses the corrected bodily fluid pressure to:
display to a physician guidance for treating the fluid management disorder; and/or
automatically control the at least one device on the intravascular catheter to relieve the fluid management disorder.

86. A method of treating fluid management disorders, the method comprising:
receiving, at a controller in a medical console, a pressure reading from within a blood vessel of a patient;
processing, by the controller, correction data to determine an offset;
calculating, by the controller using instructions stored therein and the offset, a corrected blood pressure for the blood within the vessel; and
displaying the corrected blood pressure on a display of the console or using the corrected blood pressure to control the operation of at least one device on an intravascular catheter in the blood vessel to relieve a fluid management disorder.

87. The method of claim 86, wherein:
the at least one device comprises a flow restrictor and the controller uses the corrected blood pressure to trigger activation of the restrictor; and/or
the at least one device comprises an intravascular impeller and the controller uses the corrected blood pressure to trigger activation of the impeller.

88. The method of claim 86, wherein the correction data is obtained from an ambient pressure or temperature sensor operably attached to the controller.

89. The method of claim 86, wherein the fluid management disorder is one selected from the group consisting of acute decompensated heart failure, chronic heart failure, ascites, lymphedema, chronic kidney disease, cardiac insufficiency, cardiac value regurgitation, and plural effusions.

90. The method of claim 86, wherein the controller is connected to a hub comprising one or more ports and a motor.

91. The system of claim 90, wherein the motor comprising a connection connectable to a drive shaft of an impeller pump positioned at a distal end of the intravascular catheter.

92. The method of claim 86, wherein the instructions in the controller comprise computer program instructions operable to cause the controller to activate a restrictor and/or an impeller on the catheter.

93. The method of claim 92, wherein determining the offset comprises:
reading intravascular pressure from a sensor and ambient pressure data of from a connected ambient sensor; and
calculating the offset using the intravascular pressure and the ambient pressure and optionally thermal sensitivity data and/or drift characteristic data and/or calibration data accessible to the controller.

94. The method of claim 86, further comprising using the corrected blood pressure to calculate and display a risk or degree of heart failure.

95. The method of claim 86, wherein the controller uses the corrected blood pressure to:
display to a physician guidance for treating the fluid management disorder; and/or
automatically control the at least one device on the intravascular catheter to relieve the fluid management disorder.

96. A motor control system for pumping body fluids during an intravascular fluid management procedure, the system comprises
(a) a pump assembly configured for deployment inside a patient,
(b) a motor configured to drive a pumping element of the pump assembly,
(c) a hardware controller comprising a microprocessor and a software, wherein the software is configured with the hardware controller to operate the pump assembly,
wherein the hardware controller comprises a primary control circuit, a secondary control circuit, a motor operation detection element, and a switch element,
wherein the motor operation detection element is configured to generate an electrical output proportional to an operative parameter of the motor,
wherein the switch element is configured to switch the operation of the motor from the first circuit to the second circuit when the operative parameter exceeds a predefined limit or the primary system fails.

97. The system of claim 96, wherein the pump assembly comprise a blood pump.

98. The system of claim 96, wherein the pump assembly comprise a housing encasing the pumping element.

99. The system of claim 98, wherein the pumping element comprises an impeller.

100. The system of claim 96, wherein the switch element comprises a plurality of switch states.

101. The system of claim 100, wherein the plurality of switch states comprises a first state and a second state.

102. The system of claim 101, wherein during the first state, electron flow between the primary control circuit and motor is facilitated.

103. The system of claim 101, wherein during the first state, electron flow between the primary control circuit and the motor is blocked.

104. The system of claim 101, wherein during the second state, electron flow between the secondary control circuit and motor is facilitated.

105. The system of claim 101, wherein during the first state, electron flow between the primary control circuit and the motor is blocked.

106. The system of claim 101, wherein the plurality of switch states further comprises a third state.

107. The system of claims 100 and 101, wherein switching the switch element from the first state to the second state causes the motor to operate at a preset rotation speed limit.

108. The system of claims 100 and 101, wherein switching the switch element from the first state to the second state causes the motor to operate at a preset lower rotation speed limit.

109. The system of claims 100 and 101, wherein switching the switch element from the first state to the second state causes the motor to operate at a preset standby rotation speed limit.

110. The system of claim 96, wherein the hardware controller further comprises a current detection element configured to generate an electrical output proportional to the current detection by the current detection element.

111. The system of claim 96, wherein the software provides a computational operation on the electrical output of the current detection element.

112. The system of claim 111, wherein the computational operation produces a computational output, which is used by the software to manage safe operation of the pump.

113. The system of claim 112, wherein the manage safe operation of the pump includes
(i) displaying a warning to the physician,
(ii) reducing or increasing the speed of the motor,
(iii) flushing a component of the pump assembly, and
(iv) terminating the treatment procedure.

114. The system of claim 96, wherein the hardware controller further comprises a motor torque detection element configured to generate an electrical output proportional to the torque detected by the torque detection element.

115. The system of claim 114, wherein the software provides a computational operation on the electrical output of the motor torque detection element.

116. The system of claim 115, wherein the computational operation produces a computational output, which is used by the software to manage safe operation of the pump.

117. The system of claim 116, wherein the manage safe operation of the pump includes
(i) displaying a warning to the physician,
(ii) reducing or increasing the speed of the motor,
(iii) flushing a component of the pump assembly, and
(iv) terminating the treatment procedure.

118. The system of claim 49 and its embodiments combined with the system of claim 96 and Its embodiments wherein the manage safe operation of the pump includes
(i) stopping or reducing the speed of the pump when current/torque and/or speed limits are exceeded,
(ii) running the pump at a low fixed speed when a low speed is detected or a system and/or software failure is detected.
